(19) United States
(12) Patent Application Publication
(10) Pub. No.: US 2006/0247416 A1
(43) Pub. Date: Nov. 2, 2006

(54) HIV gp41 HR2-derived synthetic peptides, and their use in therapy to inhibit transmission of human immunodeficiency virus

(76) Inventors: Mary K. Delmedico, Raleigh, NC (US); John Dwyer, Chapel Hill, NC (US)

Correspondence Address: M. Bud Nelson;Trimeris, Inc., 3500 Paramount Parkway, Morrisville, NC, 27560, US

(21) Appl. No.: 11/477,574

(22) Filed: Jun. 29, 2006

Related U.S. Application Data
(63) Continuation-in-part of application No. PCT/US04/42918, filed on Dec. 21, 2004.
(60) Provisional application No. 60/534,810, filed on Jan. 7, 2004.

(51) Int. Cl. A61K 38/16; C07K 14/16

(52) U.S. Cl. 530/324; 514/12

(57) ABSTRACT

Provided are synthetic peptides based on a native sequence of HIV gp41 HR2 except that the synthetic peptides have a plurality of amino acid replacements comprising (a) a helix-promoting amino acid, or (b) a combination of helix-promoting amino acids, and charged amino acids introduced to form ion pairs in the synthetic peptide; wherein the synthetic peptides demonstrate an unexpected, improved biological activity, as compared to a peptide having an amino acid sequence without the plurality of amino acid substitutions. Also provided are polynucleotides encoding synthetic peptide, and methods of using these synthetic peptides in inhibition of, or as compositions to inhibit, transmission of HIV to a target cell.

This is a continuation-in-part of International Application PCT/US2004/042918 with an International filing date of 21 Dec. 2004, published in English under PCT Article 21(2) and abandoned on 8 Jul. 2006; and a nonprovisional application of provisional application 60/764674.

### FIELD OF THE INVENTION

The present invention relates to synthetic peptides derived from the HR2 region of Human Immunodeficiency Virus (HIV) gp41, and their use in antiretroviral therapy as antiviral agents to inhibit transmission of HIV to target cells. More particularly, the present invention comprises a family of peptides that contain a plurality of amino acid substitutions (as compared to the native sequence) which result in unexpected, improved biological activity.

### BACKGROUND OF THE INVENTION

It is now well known that cells can be infected by HIV through a process by which fusion occurs between the cellular membrane and the viral membrane. The generally accepted model of this process is that the viral envelope glycoprotein complex (gp120/gp41) interacts with cell surface receptors on the membranes of the target cells. Following binding of gp120 to cellular receptors (e.g., CD4 in combination with a chemokine co-receptor such as CCR-5 or CXCR-4), induced is a conformational change in the gp120/gp41 complex that allows gp41 to insert into the membrane of the target cell and mediate membrane fusion.

The amino acid sequence of gp41, and its variation among different strains of HIV, is well known. FIG. 1 is a schematic representation of the generally accepted functional domains of gp41 (note the amino acid sequence numbers may vary slightly depending on the HIV strain). The fusion peptide (fusogenic domain) is believed to be involved in insertion into and disruption of the target cell membrane. The transmembrane domain, containing the transmembrane anchor sequence, is located at the C-terminal end of the protein. Between the fusion peptide and transmembrane anchor are two distinct regions, known as heptad repeat (HR) regions, each region having a plurality of heptads. The HR1 region, nearer to the N-terminal end of the protein than the HR2 region, has been generally described as comprising amino acid residues from about 545 to about 595 of the amino acid sequence of gp160. However, the amino acid numbering of gp160 depends on the strain from which the amino acid sequence was deduced. The amino acid sequence comprising the HR1 region and the amino acid sequence comprising the HR2 region are each highly conserved regions in the HIV-1 envelope protein. The HR2 region has been generally described as comprising amino acids in the positions from about 628 to about 678 of the amino acid sequence of gp160. As further shown in FIG. 1, the HR regions have a plurality of 7 amino acid residue stretches or “heptads” (the 7 amino acids in each heptad designated “a” through “g”), wherein the amino acids in the “a” position and “d” position are generally hydrophobic. Also present in each HR region is one or more leucine zipper-like motifs (also referred to as “leucine zipper-like repeats”) comprising an 8 amino acid sequence initiating with, and ending with, either an isoleucine or leucine. Most frequently, the HR2 region has just one leucine zipper like-motif, whereas the HR1 region has five leucine zipper-like motifs. These amino acid sequence features contribute to formation of a coiled coil structure of gp41, and of a coiled coil structure of peptides derived from the HR regions. Generally, coiled coils are known to be comprised of two or more helices that wrap around each other in forming oligomers, with the hallmark of coiled coils being a heptad repeat of amino acids with a predominance of hydrophobic residues at the first (“a”) and fourth (“d”) positions, charged residues frequently at the fifth (“e”) and seventh (“g”) positions, and with the amino acids in the “a” position and “d” position being primary determinants that influence the oligomeric state and strand orientation.

It was discovered that peptides derived from the native sequence of either the HR1 region (“HR1 peptides”) or HR2 region (“HR2 peptides”) of HIV gp41 inhibit transmission of HIV to host cells both in in vitro assays and in in vivo clinical studies (see, e.g., Wild et al., 1994, Proc. Natl. Acad. Sci. USA, 91:9770-9774; U.S. Pat. Nos. 5,464,933 and 5,656,480 licensed to the present assignee; and Kilby et al., 1998, Nature Med. 4:1302-1306. See also, e.g., U.S. Pat. Nos. 6,258,782 and 6,348,568 assigned to the present assignee). More particularly, HR2 peptides, as exemplified by DP178 (also known as T20, enfuvirtide, and Fuzeon®; SEQ ID NO:1), T651 (SEQ ID NO:2), T649 (SEQ ID NO:3), blocked infection of target cells with potencies of 0.5 ng/ml (EC50 against HIV-1_{LAI}; see, e.g., Lawless et al., 1996, Biochemistry, 35:13697-13708), 5 ng (IC50; HIV-1IIIB), and 2 ng (IC50; HIV-1IIIB), respectively. The respective amino acid sequences of T651 (SEQ ID NO:2) and T649 (SEQ ID NO:3) are also disclosed in U.S. Pat. No. 6,479,055 (assigned to the present assignee). Further, clinical studies have shown that treatment of an HIV-infected individual with a regimen of antiviral agents containing T20 (SEQ ID NO:1) significantly reduces the HIV-1 viral load, and significantly increases the circulating CD4⁺ cell population, in such treated individual as compared to that of an individual receiving the same regimen but without T20.

Attempts have been made to improve the biological activity of HIV-derived HR2 peptides. For example, use of an unnatural helix-favoring amino acid substitution (i.e., α-aminoisobutyric acid) in the peptide sequence, and use of chemical crosslinkers (i.e., a diaminoalkane crosslinker) each have been employed to stabilize the helical conformation of a short (14 amino acids) HIV-derived HR2 peptide of low biological activity (IC50 of >500 μM) (Sia et al., 2002, Proc. Natl. Acad. Sci. USA 99:14664-14669). Those peptides produced showed biological activity ranged only from about a 4 fold to a 15 fold increase in potency (e.g., inhibitory activity), whereas others showed no inhibitory activity (Sia et al., 2002, supra). Thus, only small gains in an already weak inhibitor (IC50 of >500 μM) were achieved by this method. Additionally, Sia et al. confirmed what is current thinking in the art; i.e., that, generally, there is a lack of correlation between helical propensity and biological activity. For example, peptides showing the highest helical content are often the weakest inhibitors of HIV-induced membrane fusion.

In another attempt to improve the biological activity of HIV-derived HR2 peptides, charged amino acids glutamic acid and lysine were substituted in various positions of the amino acid sequence of peptide “C34” in an i,i+4 arrangement (3 amino acids separating a Glu from a Lys) so that ion pairs can be formed between the Glu and Lys (in the i and the i+4 positions)(Otaka et al., 2002, Angew. Chem. Int. Ed. 41:2938-2940; see also U.S. Pat. No. 6,962,900). Substitutions ranging from 6 to 17 charged amino acids, in various combinations of Glu and Lys, were added to promote ion pair formation (i.e., shown as possibly containing between 6 and 10 i,i+4 arrangements) between Glu and Lys (see, SEQ ID NOs: 105-108). Peptides consisting of the amino acid sequences of SEQ ID NOs:105-107, with resultant possible intrahelical salt bridges, showed an increased helicity, as measured by an increase in from about 20 to about 30 percent (See, e.g., Table 2). However, peptides consisting of the amino acid sequence of SEQ ID NOs:105-108 failed to exhibit improved biological activity comprising effective antiviral potency (effective antiviral potency as measured by an IC50 of <0.50 μg/ml, and more preferably, <0.10 μg/ml) against HIV-1 strains demonstrating resistance to HR2 peptides (e.g., T20, SEQ ID NO:1), as shown in Table 2.

As has been demonstrated for other antiretroviral agents, mutations can occur in HIV during treatment which reduce the sensitivity to drug therapy using fusion inhibitor peptides such as T20 (SEQ ID NO:1). Thus, there is a need for additional compounds as fusion inhibitors which have improved biological activity. In the case of a synthetic peptide derived from the HR2 region, such improved biological activity can include, but is not limited to, about a 100 fold to about a 1,000 fold increase in inhibitory activity (as compared to a peptide consisting of the base (native) HR2 sequence from which it was derived) against HIV strains having developed resistance to known HR2 peptides, and more particularly against HIV strains that have developed resistance to any one or more of the peptides represented by SEQ ID NOs: 2-4. Additionally, typically with HR2 peptides of native (unmodified) sequence of HIV-1 gp41 (for example, a synthetic peptide, having an amino acid sequence of SEQ ID NO:2), it is difficult to achieve an injectable aqueous solution containing a synthetic peptide in a concentration of more than 100 mg/ml without encountering problems of solubility (e.g., where the formulation resembles a gel rather than a solution, or peptide precipitates out of solution over a predetermined time period) and stability (peptide being degraded over a predetermined period of time), and without adding additional components to the formulation to promote stability and/or solubility. Thus, it would also be desirable to develop an HIV fusion inhibitor peptide having improved solubility and stability, while also having improved pharmacological properties.

Thus, there is a need for an HIV fusion inhibitor peptide which, as compared to the base sequence of any one of SEQ ID NOs: 2-4, demonstrates improved biological activity such as demonstrated by one or more of improved antiviral activity against HIV-1 strains resistant to HR2 peptides, improved solubility and stability in an aqueous solution, and improved pharmacological properties. The present invention addresses these needs.

### SUMMARY OF THE INVENTION

The present invention relates to synthetic peptides derived from a base amino acid sequence (“base sequence”) of one or more of SEQ ID NO:2, SEQ ID NO:3 or SEQ ID NO:4, wherein the synthetic peptide was made to differ from the base sequence by adding a plurality of amino acids in the amino acid sequence of the synthetic peptide in place of amino acids within the amino acid sequence of the base sequence, wherein such synthetic peptide demonstrates an unexpected, improved biological activity, as compared to a peptide having the base sequence of one or more of SEQ ID NO:2 or SEQ ID NO:3 or SEQ ID NO:4.

Provided are synthetic peptides derived from the HR2 region of gp41, wherein each such synthetic peptide comprises a base sequence of one or more of SEQ ID NO:2 or SEQ ID NO:3 or SEQ ID NO:4, except that the amino acid sequence of the synthetic peptide differs from that of the base sequence by: comprising a plurality of amino acid substitutions within the amino acid sequence of the base sequence using one or more helix-promoting amino acids; and demonstrating an unexpected, improved biological activity as compared to a peptide having a base sequence of one or more of SEQ ID NO:2 or SEQ ID NO:3 or SEQ ID NO:4. The plurality of amino acid substitutions using a helix-promoting amino acid can range from about 5 to about 15 amino acids, depending on the length of the base sequence from which the synthetic peptide was derived; and more particularly from about 5% to about 50% of the amino acids within a base sequence of one or more of SEQ ID NO:2 or SEQ ID NO:3 or SEQ ID NO:4 may be replaced with one or more helix-promoting amino acids to produce a synthetic peptide according to the present invention. In a preferred embodiment, the synthetic peptide differs from a base sequence of one or more of SEQ ID NO:2 or SEQ ID NO:3 or SEQ ID NO:4 in that, as a result of the substitutions with a helix promoting amino acid, the synthetic peptide has an amino acid sequence having not less than 2 and not more than 5 leucine zipper-like motifs (as compared to one leucine zipper like motif in a base sequence of one or more of SEQ ID NO:2 or SEQ ID NO:3 or SEQ ID NO:4).

Provided is a synthetic peptide comprising a base sequence of one or more of SEQ ID NO:2 or SEQ ID NO:3 or SEQ ID NO:4, except that the synthetic peptide differs from the base sequence by: (a) adding amino acids in the production of the synthetic peptide which substitute for amino acids in corresponding positions within the amino acid sequence of the base sequence, wherein the added amino acids consist essentially of (i) a plurality of amino acid substitutions comprising one or more helix-promoting amino acids, and (ii) a plurality of charged amino acids which are spaced apart, in the amino acid sequence of the synthetic peptide, from oppositely charged amino acids in forming a plurality of ion pairs (preferably, either in an i, i+4 arrangement and/or i,i+3 arrangement) in the synthetic peptide; and (b) demonstrating an improved biological activity as compared to a base sequence of one or more of SEQ ID NO:2 or SEQ ID NO:3 or SEQ ID NO:4. Preferably, the number of ion pairs in the synthetic peptide range from about 3 to about 10. Preferably, the synthetic peptide further comprises an increase in helicity, as compared to a base sequence of one or more of SEQ ID NO:2 or SEQ ID NO:3 or SEQ ID NO:4. The plurality of amino acid substitutions comprising one or more helix promoting amino acids and a plurality of charged amino acids can range from about 5 to about 25 amino acids, depending on the length of the base sequence from which the synthetic peptide is derived; and more particularly, from about 5% to about 60% of the amino acids within the amino acid sequence of a base sequence of one or more of SEQ ID NO:2 or SEQ ID NO:3 or SEQ ID NO:4 may be substituted with a combination of helix-promoting amino acids and charged amino acids to produce a synthetic peptide according to the present invention. In a preferred embodiment, the synthetic peptide differs from a base sequence of one or more of SEQ ID NO:2 or SEQ ID NO:3 or SEQ ID NO:4 in that, as a result of the substitutions with a helix promoting amino acid, the synthetic peptide has an amino acid sequence having not less than 2 and not more than 5 leucine zipper-like motifs.

Provided are synthetic peptides derived from a base sequence of one or more of SEQ ID NO:2 or SEQ ID NO:3 or SEQ ID NO:4, wherein the synthetic peptide has a plurality of amino acids in it's amino acid sequence which differs from, and replaces amino acids within, the amino acid sequence of a base sequence of one or more of SEQ ID NO:2 or SEQ ID NO:3 or SEQ ID NO:4, the plurality of amino acids comprising one or more of:. (a) a helix-promoting amino acid; and (b) a plurality of charged (polar) amino acids introduced to form ion pairs (preferably, in either an i,i+3 arrangement and/or i,i+4 arrangement) between oppositely charged amino acids; wherein the synthetic peptide has an improved biological activity, as compared to a base sequence of any one or more of SEQ ID NO:2 or SEQ ID NO:3 or SEQ ID NO:4. The synthetic peptide may further comprise one or more or an increase in helicity, as compared to a peptide having a base sequence of one or more of SEQ ID NO:2 or SEQ ID NO:3 or SEQ ID NO:4; and a stability, as measured by a Tm (melting temperature), in the range of from about 36° C. to about 75° C. The plurality of amino acid substitutions, made within a base sequence in producing a synthetic peptide, comprising a helix-promoting amino acid and a charged amino acid can range from about 5 to about 25 amino acids, depending on the length of the base sequence; and more particularly from about 5% to about 60% of the amino acids within the base sequence may be substituted with a combination comprising a helix-promoting amino acid and a charged amino acid to produce a synthetic peptide according to the present invention. In a preferred embodiment, the synthetic peptide differs from a base sequence of one or more of SEQ ID NO:2 or SEQ ID NO:3 or SEQ ID NO:4 in that, as a result of the substitutions with a helix promoting amino acid, the synthetic peptide has an amino acid sequence having not less than 2 and not more than 5 leucine zipper-like motifs.

Unexpectedly, a synthetic peptide according to the present invention demonstrates improved biological activity as compared to a peptide having a base sequence of one or more of SEQ ID NO:2 or SEQ ID NO:3 or SEQ ID NO:4. Preferably, the biological activity that is improved comprises one or more biological properties selected from the group consisting of biological half-life (e.g., enabling the synthetic peptide to survive longer in vivo before being degraded in and/or removed from the bloodstream), solubility in an aqueous solution, stability in an aqueous solution, more potency (more potent antiviral activity) against HIV strains that have developed resistance to peptides derived from the natural sequence of HIV gp41 HR2 region (e.g., a peptide having the amino acid sequence of SEQ ID NO:2), and a combination thereof.

A synthetic peptide according to the invention may further comprise an N-terminal group, a C-terminal group, or both an N-terminal group and C-terminal group, as described in more detail. Also provided is a pharmaceutical composition or medicament comprising the synthetic peptide according to the present invention and at least one additional component comprising a pharmaceutically acceptable carrier, a macromolecule, or a combination thereof.

Provided is the use of a synthetic peptide according to the present invention as an active therapeutic substance in therapy of HIV infection. Also provided, is the use of a synthetic peptide according to the present invention for the manufacture of a medicament for a therapeutic application comprising treatment of HIV. The present invention also provides methods of using a synthetic peptide according to the present invention. In one embodiment, a synthetic peptide is used as a part of a therapeutic regimen containing one or more additional antiviral agents. Such therapeutic regimen is used for the therapy of HIV infection.

In another embodiment, provided are methods for using the synthetic peptide according to the present invention to treat HIV-infected individuals. In one example, a method of using a synthetic peptide according to the present invention for inhibition of transmission of HIV to a target cell comprises adding to the virus and the cell an amount of a synthetic peptide according to the present invention effective to inhibit infection of the cell by the virus. Also, according to the present invention, provided is a method for inhibition of transmission of HIV to a cell, comprising contacting the virus in the presence of a cell with an amount of synthetic peptide according to the present invention effective to inhibit infection of the cell by HIV. Also provided is a method for inhibiting HIV fusion (e.g., a process by which HIV gp41 mediates fusion between the viral membrane and cell membrane during infection by HIV of a target cell), comprising contacting the virus in the presence of a cell with a concentration of synthetic peptide according to the present invention effective to inhibit HIV membrane fusion.

The above, and other features and advantages of the present invention, will be apparent in the following Detailed Description of the Invention when read in conjugation with accompanying drawings.

### BRIEF DESCRIPTION OF THE DRAWINGS

FIG. 1 is a schematic of HIV gp41 showing the heptad repeat 1 region (HR1) and heptad repeat 2 region (HR2) along with other functional regions of gp41. Exemplary peptide sequences corresponding to HIV_{LAI} HR1 and HR2 are shown for purposes of illustration. The amino acid residues are numbered according to their position in gp160, train HIV_{LAI}.

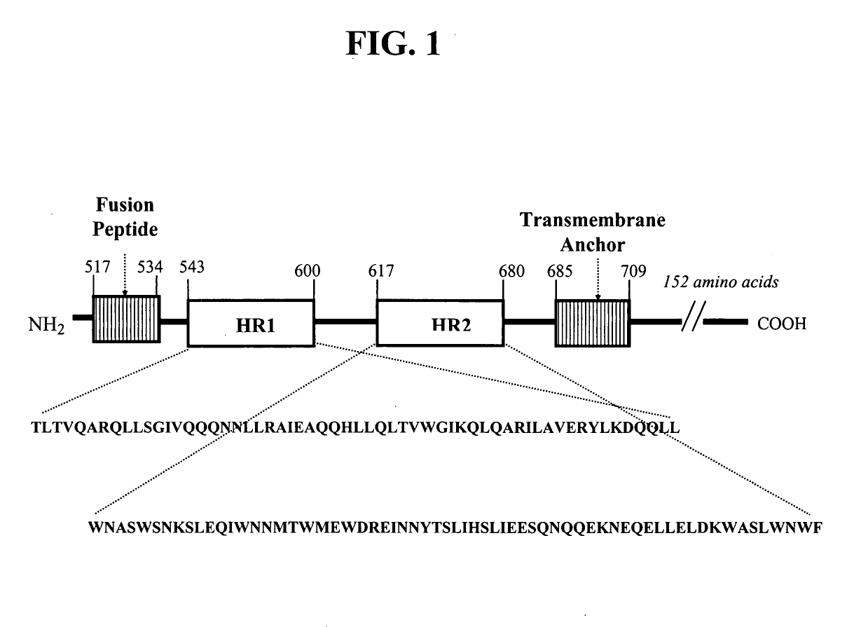

FIG. 2 shows a comparison of polymorphisms contained within SEQ ID NO:2 of the HR2 region of HIV gp41 as determined from various laboratory strains and clinical isolates, wherein variations in amino acid sequence are indicated by the single letter amino acid code.

### DETAILED DESCRIPTION OF THE INVENTION

Definitions:

The term “individual”, when used herein for purposes of the specification and claims, means a mammal, and preferably a human.

The term “target cell”, when used herein for purposes of the specification and claims, means a cell capable of being infected by HIV. Preferably, the cell is a human cell or are human cells; and more preferably, human cells capable of being infected by HIV via a process including membrane fusion.

The term “pharmaceutically acceptable carrier”, when used herein for purposes of the specification and claims, means a carrier medium that does not significantly alter the biological activity of the active ingredient (e.g., a synthetic peptide according to the present invention) to which it is added. As known to those skilled in the art, a suitable pharmaceutically acceptable carrier may comprise one or substances, including but not limited to, water, buffered water, saline, 0.3% glycine, aqueous alcohols, isotonic aqueous buffer; and may further include one or more substances such as water-soluble polymer, glycerol, polyethylene glycol, glycerin, oils, salts such as sodium, potassium, magnesium and ammonium, phosphonates, carbonate esters, fatty acids, saccharides, polysaccharides, glycoproteins (for enhanced stability), excipients, and preservatives and/or stabilizers (to increase shelf-life or as necessary and suitable for manufacture and distribution of the composition). Preferably, the carrier is suitable for intravenous, intramuscular, subcutaneous or parenteral administration.

By the term “amino acid” is meant, for purposes of the specification and claims and in reference to the synthetic peptides according to the present invention, to refer to a molecule that has at least one free amine group and at least one free carboxyl group. The amino acid may have more than one free amine group, or more than one free carboxyl group, or may further comprise one or more free chemical reactive groups other than an amine or a carboxyl group (e.g., a hydroxyl, a sulfhydryl, etc.). The amino acid may be a naturally occurring amino acid (e.g., L-amino acid), a non-naturally occurring amino acid (e.g., D-amino acid), a synthetic amino acid, a modified amino acid, an amino acid derivative, an amino acid precursor, and a conservative substitution. One skilled in the art would know that the choice of amino acids incorporated into a peptide will depend, in part, on the specific physical, chemical or biological characteristics required of the antiviral peptide. Such characteristics are determined, in part, by determination of helicity (as described herein in more detail) and antiviral activity (as described herein in more detail). For example, the skilled artisan would know from the descriptions herein that amino acids in a synthetic peptide may be comprised of one or more of naturally occurring (L)-amino acid and non-naturally occurring (D)-amino acid. A preferred amino acid may be used to the exclusion of amino acids other than the preferred amino acid. By the term “an amino acid comprising isoleucine or leucine”, unless otherwise specifically pointed out, is meant for purposes of the specification and claims and in reference to a helix promoting amino acid added to synthetic peptide according to the present invention (in substituting for an amino acid within the base sequence), to refer to isoleucine or leucine, respectively, or their respective naturally occurring amino acid (e.g., L-amino acid), non-naturally occurring amino acid (e.g., D-amino acid), isomeric form (e.g., norleucine, allo-isoleucine, and the like) or a derivative form (e.g., tert-leucine). A preferred form of an amino acid may be used to the exclusion of forms of the amino acid other than the preferred form of the amino acid.

A “helix-promoting amino acid” is meant, for purposes of the specification and claims, to refer to an amino acid that is a nonpolar amino acid and that has a high propensity to promote alpha helix formation of an amino acid sequence containing such helix-promoting amino acid. It is known in the art that naturally occurring amino acids which are helix-promoting amino acids include alanine, leucine, methionine, glycine, isoleucine, phenylalanine, valine, and trytophan, and non-naturally occurring amino acids such as an amino-butyric acid (e.g., α-aminoisobutyric acid). In terms of these helix-promoting amino acids, the order of helical propensity (greatest/higher to lesser/lower) is: alanine, leucine, methionine, phenylalanine, isoleucine, and trytophan. Thus, in accordance with the present invention, a synthetic peptide comprises a plurality of amino acid substitutions comprising a helix-promoting amino acid in a position in the amino acid sequence of the synthetic peptide, which amino acid has a higher helical propensity as compared to the amino acid in a corresponding position of amino acid sequence of the base sequence which is substituted by the helix-promoting amino acid. In another embodiment, a helix-promoting amino acid (which is not charged), is substituted for a charged amino acid within the base sequence. The term “helix-promoting”, when used herein for purposes of the specification and claims, is customarily referring to the effect of one or more amino acid substitutions on contributing to the helicity of a peptide; and more particularly, an effect observed as one or more of alpha helix stabilizing, or increase in helicity, as known in the art.

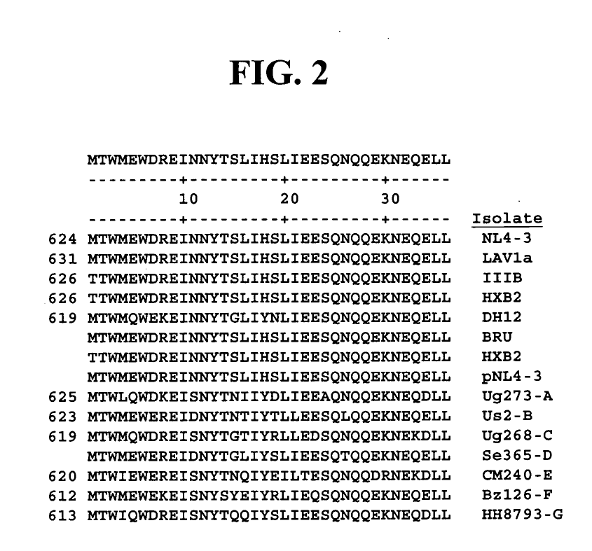

A “conservative substitution”, in relation to amino acid sequence of a synthetic peptide according to the present invention, is a term used hereinafter for the purposes of the specification and claims to mean one or more amino acids substitution in the sequence of the synthetic peptide such that the synthetic peptide still demonstrates (conserved is) the unexpected, improved biological activity, as described in more detail herein. As known in the art “conservative substitution” is defined by aforementioned function, and includes substitutions of amino acids having substantially the same charge, size, hydrophilicity, and/or aromaticity as the amino acid replaced. Such substitutions are known to those of ordinary skill in the art to include, but are not limited to, glycine-alanine-valine; isoleucine-leucine; tryptophan-tyrosine; aspartic acid-glutamic acid; arginine-lysine; asparagine-glutamine; and serine-threonine. Such substitutions may also comprise polymorphisms, as illustrated in FIG. 2, at the various amino acid positions in SEQ ID NO:2 as found in laboratory, various clades, and/or clinical isolates of HIV.

The term “HIV” refers to Human Immunodeficiency Virus, and more preferably HIV-1.

The term “native sequence”, when used herein for purposes of the specification and claims and in reference to the amino acid sequence of the HR2 region of HIV gp41, means a naturally occurring sequence found in laboratory HIV strains and/or HIV clinical isolates. Such sequences are readily available from public gene databases such as GenBank. For purposes of illustration, but not limitation, some of such native sequences are illustrated in FIG. 2, in which illustrative substitutions (e.g., polymorphisms) are noted in various amino acid positions in the amino acid sequence represented by SEQ ID NO:2. The term “base sequence”, when used herein for purposes of the specification and claims, refers to the amino acid sequence (or a peptide consisting of the amino acid sequence) of any one or more of SEQ ID NO:2 or SEQ ID NO:3 or SEQ ID NO:4 (which include polymorphisms found in the native sequence of the corresponding portion of the HR2 region of HIV gp421, as shown for example in FIG. 2). A base sequence is used in the design of a synthetic peptide according to the present invention, and hence the synthetic peptide is “derived” from a base sequence. Thus, for example, a synthetic peptide is derived from the base sequence in that the synthetic peptide comprises some of the amino acid sequence of the base sequence, in addition to differing from the base sequence by replacing amino acids within the base sequence with either (a) a plurality of helix-promoting amino acids into the amino acid sequence of the synthetic peptide, or (b) a combination of helix-promoting amino acids, and charged amino acids (to form ion pairs) in forming a synthetic peptide having unexpected, improved biological activity, as compared to a peptide having a base sequence of any one or more of SEQ ID NO:2 or SEQ ID NO:3 or SEQ ID NO:4.

The term “reactive functionality”, when used herein for purposes of the specification and claims, means a chemical group or chemical moiety that is capable of forming a covalent bond and/or is protective (e.g., protects peptide from reacting with themselves or other molecules). With respect to chemical groups, a reactive functionality is known to those skilled in the art to comprise a group that includes, but is not limited to, maleimide, thiol, carboxy, phosphoryl, acyl, hydroxyl, acetyl, hydrophobic, amido, dansyl, fluorenylmethyoxycarbonyl (Fmoc), t-butyloxycarbonyl (Boc), sulfo, a succinimide, a thiol-reactive, an amino-reactive, a carboxyl-reactive, and the like. For example, a chemical group, added to the N-terminal amino acid of a synthetic peptide to block chemical reactivity of that amino terminus of the peptide, comprises an N-terminal group. Such N-terminal groups for protecting the amino terminus of a peptide are well known in the art, and include, but are not limited to, lower alkanoyl groups, acyl groups, sulfonyl groups, and carbamate forming groups. Preferred N-terminal groups may include acetyl, Fmoc, and Boc. For example, a chemical group, added to the C-terminal amino acid of a synthetic peptide to block chemical reactivity of that carboxy terminus of the peptide, comprises a C-terminal group. Such C-terminal groups for protecting the carboxy terminus of a peptide are well known in the art, and include, but are not limited to, an ester or amide group. A chemical moiety may comprise a linker. Linkers are known to refer to a compound or moiety that acts as a molecular bridge to operably link two different molecules (e.g., a wherein one portion of the linker binds to a peptide according to the present invention, and wherein another portion of the linker binds to a macromolecular carrier or another antiviral peptide known to inhibit HIV transmission to a target cell). The two different molecules may be linked to the linker in a step-wise manner. There is no particular size or content limitations for the linker so long as it can fulfill its purpose as a molecular bridge. Linkers are known to those skilled in the art to include, but are not limited to, chemical chains, chemical compounds (e.g., reagents), and the like. The linkers may include, but are not limited to, homobifunctional linkers and heterobifunctional linkers. Heterobifunctional linkers, well known to those skilled in the art, contain one end having a first reactive functionality to specifically link a first molecule, and an opposite end having a second reactive functionality to specifically link to a second molecule. It will be evident to those skilled in the art that a variety of bifunctional or polyfunctional reagents, both homo- and hetero-functional (such as those described in the catalog of the Pierce Chemical Co., Rockford, Ill.) or a maleimide, may be employed as a linker with respect to the present invention. Depending on such factors as the molecules to be linked, and the conditions in which the linking is performed, the linker may vary in length and composition for optimizing such properties as preservation of biological function stability, resistance to certain chemical and/or temperature parameters, and of sufficient stereo-selectivity or size. For example, the linker should not significantly interfere with the ability of the peptide according to the present invention (to which it is linked) to function as an inhibitor of either or both of HIV fusion and HIV transmission to a target cell. A preferred reactive functionality may be used to the exclusion of reactive functionalities other than the preferred reactive functionality.

The term “macromolecular carrier”, when used herein for purposes of the specification and claims, means a molecule which is linked, joined, or fused (e.g., chemically or through recombinant means) to one or more peptides according to the present invention, whereby the molecule is capable of conferring one or more of stability to the one or more peptides, increase in biological activity of the one or more peptides, or increase in serum half-life of the one or more peptides (e.g., prolonging the persistence of the one or more peptides in the body) relative to that with respect to the one or more peptides in the absence of the molecule. Such macromolecular carriers are well known in the art to include, but are not limited to, serum proteins, polymers, carbohydrates, and lipid-fatty acid conjugates. Serum proteins typically used as macromolecular carriers include, but are not limited to, transferrin, albumin (preferably human), immunoglobulins (preferably human IgG or one or more chains thereof), or hormones. Polymers typically used as macromolecular carriers include, but are not limited to, polylysines or poly(D-L-alanine)-poly(L-lysine)s, or polyols. A preferred polyol comprises a water-soluble poly(alkylene oxide) polymer, and can have a linear or branched chain. Suitable polyols include, but are not limited to, polyethylene glycol (PEG), polypropylene glycol (PPG), and PEG-PPG copolymers. A preferred polyol comprises PEG having an average molecular size selected from the range of from about 1,000 Daltons to about 20,000 Daltons. Other types of macromolecular carriers that can be used, which generally have molecular weights higher than 20,000, are known in the art.

The term “synthetic”, in relation to a peptide according to the present invention, is used hereinafter for the purposes of the specification and claims to mean that the peptide is produced by chemical synthesis, recombinant expression, biochemical or enzymatic fragmentation of a larger molecule, chemical cleavage of larger molecule, a combination of the foregoing or, in general, made by any other method in the art, and isolated. The term “isolated” when used in reference to a peptide, means that the synthetic peptide is substantially free of components which have not become part of the integral structure of the peptide itself; e.g., such as substantially free of cellular material or culture medium when produced by recombinant techniques, or substantially free of chemical precursors or other chemicals when chemically synthesized or produced using biochemical or chemical processes.

The term “ion pair”, when used herein for purposes of the specification and claims, is customarily referring to a simple electrostatic interaction between oppositely charged ions (e.g., between two oppositely charged amino acids) in an amino acid sequence. Each oppositely charged ion is on a side chain of an amino acid. Of the different types of ion pairs, a “salt bridge” is an ion pair in close spatial relationship (as known in the art), as determined by nuclear magnetic resonance or other standard method known in the art. In a preferred embodiment, an ion pair is formed by 2 oppositely charged amino acid residues spaced apart by either three amino acids (i.e., in an i,i+4 arrangement) or two amino acids (i.e., in an i,i+3 arrangement) in a contiguous sequence contained in an amino acid sequence in forming a helix. Thus, a positively charged amino acid (e.g., lysine, arginine, histidine) may form an ion pair with a negatively charged amino acid (e.g., glutamic acid, aspartic acid). Thus, for example, in one embodiment, a synthetic peptide is derived from a base sequence except that in the place of a neutral (charge) or negatively charged amino acid (as in the base sequence) included in a corresponding position in the synthetic peptide is a positively charged amino acid placed such that an ion pair is formed (e.g., in an i,i+3 arrangement or i,i+4 arrangement) with a negatively charged amino acid. In another embodiment, a synthetic peptide is derived from a base sequence except that in the place of a neutral (charge) or positively charged amino acid (as in the base sequence), included in the synthetic peptide is a negatively charged amino acid in a position in the amino acid sequence such that an ion pair is formed (e.g., in an i,i+3 arrangement or i,i+4 arrangement) with a positively charged amino acid. In yet another embodiment, both a negatively charged amino acid and a positively charged amino acid are included in the synthetic peptide in an arrangement such that an ion pair is formed in the synthetic peptide, which ion pair is absent in the base sequence from which the synthetic peptide is derived.

The term “improved biological activity”, when used herein for purposes of the specification and claims in reference to a synthetic peptide according to the present invention, means that (a) increased (more potent) is the antiviral activity (e.g., as measured by the IC50 or other measurement standard in the art for measuring antiviral potency) of a synthetic peptide, as compared to the antiviral activity of a peptide having a base sequence of SEQ ID NO:2 or SEQ ID NO:3 or SEQ ID NO:4, against HIV strains showing reduced susceptibility (“resistance”) to the antiviral activity of the base sequence; or (b) increased is the antiviral activity of a synthetic peptide, as compared to the antiviral activity of a base sequence of SEQ ID NO:2 or SEQ ID NO:3 or SEQ ID NO:4, against HIV strains showing resistance to the antiviral activity of the base sequence (see, Tables 1 and 2, “HIV-RY”); and improved are the pharmacokinetic properties (e.g., as measured by one or more parameters such as Area Under the Curve (AUC), biological half-life, and or clearance; or other measurement standard in the art for measuring pharmacokinetic properties) of a synthetic peptide, as compared to the pharmacokinetic properties of a peptide having a base sequence of SEQ ID NO:2 or SEQ ID NO:3 or SEQ ID NO:4. It is believed that such improved biological activity is unexpected. In one embodiment, improved biological activity comprises an increase in antiviral activity preferably no less than 20 fold more potent activity, as that observed for a peptide having the base sequence and in relation to virus isolates (mutants) which are resistant to the base sequence. In a more preferred embodiment, such improved biological activity comprises an IC50 of less than or equal to 0.500 μg/ml against virus isolates resistant to a peptide of the base sequence. More preferably, such IC50 of the synthetic peptide is in the nanogram/ml or picogram/ml range. In a preferred embodiment of the present invention, a peptide of the base sequence consists of an amino acid sequence selected from the group consisting of SEQ ID NO:2, SEQ ID NO:3, SEQ ID NO:4, and a combination thereof. It is important to note that the improved biological activity of synthetic peptides against virus isolates resistant to a peptide having the base sequence can also correlate to unexpected and improved antiviral activity against isolates HIV resistant to T20 (SEQ ID NO:1). A synthetic peptide has improved pharmacokinetic properties when the synthetic peptide has one or more of (a) a longer biological half life (t ½), and (b) a reduction in biological clearance (Cl); as compared to that of a peptide of the base sequence selected from the group consisting of SEQ ID NO:2, SEQ ID NO:3, SEQ ID NO:4, and a combination thereof. In a preferred embodiment, the synthetic peptide typically allows for a clearance that is reduced by no less than 30 percent relative to that of a peptide of the base sequence from which it is derived, as will be shown in more detail in the examples herein. In another preferred embodiment, the synthetic peptide typically allows for an increase in biological half-life of no less than 5 fold as compared to the biological half-life of a peptide of the base sequence from which it is derived, as will be shown in more detail in the examples herein. The term “improved biological activity” may additionally comprise a greater stability in solution demonstrated by the synthetic peptide, as compared to the stability in solution of a peptide having the native sequence of gp41 (e.g., a peptide having the amino acid sequence of SEQ ID NO:1), as will be described in more detail herein.

The term “pharmacokinetic properties”, when used herein for purposes of the specification and claims, means the total amount of active ingredient (e.g., synthetic peptide analog) in a pharmaceutical composition that is systematically available over time. Pharmacokinetic properties may be determined by measuring total systemic concentrations of synthetic peptide analog over time after administration, either singularly or in comparison with pharmacokinetic properties after administration of synthetic peptide alone (i.e., with no amide-forming amino acid operably bound thereto). As an example, pharmacokinetic properties may be expressed in terms of the Area Under the Curve (AUC), biological half-life, and/or clearance. AUC is the integrated measure of systemic active ingredient concentrations over time, in units of mass x time/volume. Following the administration of a dose of active ingredient, the AUC from the time of dosing to the time when no active ingredient remains in the body, is a measure of the exposure of the individual to the active ingredient (and/or a metabolite of an active ingredient). Clearance is defined as dose/AUC, and is expressed in units of volume/weight/time.

The term “in solution”, as standard in the art in referring to an aqueous fluid into which is dissolved one or more solids, is used herein for the purposes of the specification and claims to mean an aqueous solution containing the peptide dissolved therein under realistic use conditions of concentration and temperature as described herein in more detail and as standard in the art for an injectable drug formulation. There are various ways known in the art to distinguish formation of a solution, as opposed to formation of a suspension, such as checking for visual clarity (transparency of a solution versus cloudiness of a suspension), light transmission, and the like. “Solubility” is determined by the amount (e.g., weight percent) of HIV fusion inhibitor peptide that is present in solution in an aqueous fluid without showing observed evidence of precipitation out of solution, or gelling of the aqueous fluid containing the HIV fusion inhibitor peptide. “Stability”, in referring to a peptide in solution, is determined by the amount of HIV fusion inhibitor peptide, in solution, that degrades over time.

The term “stability”, when used herein for purposes of the specification and claims in reference to the structure of a synthetic peptide according to the present invention, means the stability of the alpha-helical coiled coil structure of the peptide. It is known by those skilled in the art that stability can be measured by standard methods known in the art, such as by determining the melting temperature (“Tm”) of the peptide (see, e.g., Example 1 herein). In a preferred embodiment, a synthetic peptide, comprising a plurality of amino acid substitutions as described herein, and as compared to a base sequence from which the synthetic peptide is derived, demonstrates greater stability as may be discerned by observing a higher melting temperature of the synthetic peptide, as compared to the melting temperature of the base sequence from which the synthetic peptide was derived. Such one or more amino acid substitutions may include introduction of helix-promoting amino acids in a proper position (e.g., in replacing side chains of lower helix propensity); or introduction helix-promoting amino acids, and charged amino acids (in forming ion pairs), in one or more heptad repeats that serve to stabilize the coiled coil.

The term “percent identity”, when used herein for purposes of the specification and claims in reference to a sequence according to the present invention, means that the sequence is compared (“Compared Sequence”) to a described or reference sequence (“Reference Sequence”); wherein a percent identity is determined according to the following formula:
percent identity=[1−(xC/yR)]×100
wherein xC is the number of differences between the Reference Sequence and the Compared Sequence over the length of alignment between the Compared Sequence and Reference Sequence wherein (a) each base or amino acid in the Reference Sequence that does not have a corresponding aligned base or amino acid compared to the Compared Sequence, and (b) each gap in the Reference Sequence, and (c) each aligned base or amino acid in the Compared Sequence that is different from an aligned base or amino acid in the Reference Sequence, constitutes a difference; and yR is the number of bases or amino acids in the Reference Sequence over the length of the Compared Sequence with any gap created in the Reference Sequence as a result of alignment also being counted as a base or amino acid. Methods and software for alignment between two predetermined sequences are well known in the art. Thus, for example, a Reference Sequence may be a synthetic peptide having an amino acid sequence of any one of SEQ ID NOs: 5-103, and a Compared Sequence is a synthetic peptide which is compared to the Reference Sequence for percent identity. In one embodiment, the amino acid sequence of any one of SEQ ID NOs: 5-103 has at least 90% identity with the Compared Sequence. In another embodiment, the amino acid sequence of any one of SEQ ID NOs: 5-103 has at least 95% identity with the Compared Sequence.

The terms “treatment” or “therapy”, are used interchangeably with respect to HIV infection, and for purposes of the specification and claims, to mean that a synthetic peptide (or a composition having the synthetic peptide as an active drug substance) may be used to affect one or more processes associated with HIV infection, or one or more parameters or endpoints used as indicators for determining the therapeutic effect of such treatment or therapy (e.g., “therapeutic application”). For example, the synthetic peptide may be used to inhibit one or more of the following processes: transmission of HIV to a target cell; fusion between HIV and a target cell (“HIV fusion”); viral entry (the process of HIV or its genetic material entering into a target cell during the infection process); and syncytia formation (e.g., between an HIV-infected cell, and a target cell). Viral suppression (determined by methods known in the art for measuring the viral load of HIV in a body fluid or tissue) is a commonly used primary endpoint, and an increase in the number of CD4⁺ cells circulating in the bloodstream is a commonly used secondary endpoint, for assessing the efficacy of a drug in treatment or therapy of HIV infection; each being a measurable effect of inhibiting transmission of HIV to a target cell. Thus, a synthetic peptide may be used to in a method of suppressing the viral load (amount of HIV) in an HIV-infected individual by administering the synthetic peptide to the individual in an amount effective for suppressing the viral load; and in a method of increasing the relative number of circulating CD4⁺ cells (as compared to the relative number of circulating CD4⁺ cells before treatment with synthetic peptide) in an HIV-infected individual by administering the synthetic peptide to the individual in an amount effective for increasing the relative number of circulating CD4⁺ cells.

[End of formal definition section]

A synthetic peptide of the present invention comprises the following distinguishing and functional characteristics.

A. Sequence.

A synthetic peptide according to the present invention is derived from the native sequence of the HR2 region of HIV-1 gp41, and more particularly comprises any one or more of SEQ ID NO:s 2, 3, or 4 as a base sequence; however, the synthetic peptide differs from the base sequence by the inclusion in the amino acid sequence of the synthetic peptide of a plurality of amino acid substitutions (as compared to the corresponding positions within the base sequence) which result in an unexpected, improved biological activity, and may further comprise an increase in helicity of the synthetic peptide, as compared to the helicity of a peptide of the base sequence. In one embodiment, the synthetic peptide differs from the base sequence by: (a) by substitution of between about 5% and about 50% of the amino acids within the base sequence with a helix-promoting amino acid (e.g., adding an amino acid having a greater helical propensity in place of an amino acid having a lower helical propensity than the amino acid replacing it, or adding a helix-promoting amino acid in place of a charged amino acid); and (b) comprising improved biological activity as compared to a peptide of the base sequence. Such synthetic peptides are exemplified by a synthetic peptide having an amino acid sequence of SEQ ID NO:5, or an amino acid sequence having at least 90% identity with SEQ ID NO:5 and differing from the base sequence by (i) an addition of a plurality of helix-promoting amino acids as compared to corresponding amino acid positions within the base sequence from which the synthetic peptide is derived, and (ii) improved biological activity as compared to a peptide of a base sequence of any one of SEQ ID NOs: 2, 3, or 4. In another embodiment, the synthetic peptide has two or more substitutions that include at least an “a” position in one heptad and a “d” position in a different (preferably adjacent) heptad surprisingly resulting in improved biological activity, as compared to a peptide of the base sequence of any one of SEQ ID NOs: 2, 3, or 4. More preferably, the total number of “a” and “d” positions of the base sequence which are substituted with a helix-promoting amino acid ranges from 2 to 5. In a preferred embodiment, the helix-promoting amino acid is either a leucine or isoleucine, or a combination of leucine and isoleucine, in forming from 1 to 3 additional leucine zipper-like motifs as compared to the base sequence of any one of SEQ ID NOs: 2, 3, or 4. Such synthetic peptides are exemplified by SEQ ID NOs:82, 84, 85, 86, 87, 97, 98, 99, 100, 101, 102, and 103.

In another embodiment, the synthetic peptide differs from the base sequence of any one of SEQ ID NOs: 2, 3, or 4 by (a) substitution of between about 5% and about 60% of the amino acids within the base sequence with (i) a helix-promoting amino acid, and (ii) a charged amino acid residue, resulting in formation of a plurality of ion pairs in the synthetic peptide which were not present in the base sequence, and more preferably in an arrangement wherein the synthetic peptide comprises a number of ion pairs ranging from about 3 ion pairs to about 10 ion pairs; and (b) comprising improved biological activity as compared to a peptide of the base sequence. Such a synthetic peptide is exemplified by a synthetic peptide having an amino acid sequence selected from the group consisting of SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:83, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, and SEQ ID NO:103; or an amino acid sequence having at least 90% identity with any one or more of SEQ ID NOs:6-81, 83, and 88-103, and differing from a base sequence of any one or more of SEQ ID NOs: 2, 3, or 4 by (i) an addition of a plurality of helix-promoting amino acids as compared to corresponding amino acid positions within the base sequence, (ii) an addition of a plurality of charged amino acids as compared to corresponding amino acid positions within the base sequence, and (iii) improved biological activity as compared to a peptide having the base sequence. A synthetic peptide according to the present invention is not a peptide consisting of an amino acid sequence of any one of SEQ ID NOs:105-108, which peptides reflect changes consisting of insertion of charged amino acids into native sequence. A preferred synthetic peptide may be used to the exclusion of synthetic peptide other than the preferred synthetic peptide.

In another embodiment, in addition to the base sequence within which substitutions are made to produce a synthetic peptide according to the present invention as described above, the synthetic peptide may further comprise one or more of: an additional 1 to about 20 amino acids added at its N-terminus (e.g., sequence additional to and typically different from the base sequence of any one or more of SEQ ID NOs: 2, 3, or 4); a deletion of from about 1 to 10 amino acids from the N-terminus (e.g., N-terminal end and inward) of the base sequence from which the synthetic peptide is derived; an additional 1 to about 20 amino acids at the C-terminus of the synthetic peptide (e.g., sequence additional to and typically different from the base sequence of any one or more of SEQ ID NOs: 2, 3, or 4); and a deletion of from about 1 to 10 amino acids from the C-terminus (e.g., C-terminal end and inward) of the base sequence from which the synthetic peptide is derived. Illustrations of this embodiment include, but are not limited to, SEQ ID NOs: 14-23, 37-49, 63, 64, 66, 68, 69, 72-75, 78, 80, and 88.

For purposes of illustrating the invention, base sequences (SEQ ID NOs. 2, 3, and 4) share the following amino acid sequence (SEQ ID NO:3).

WMEWDREINNYTSLIHSLIEESQNQQEKNEQELLEL

In one embodiment, a synthetic peptide comprises, as compared to a base sequence from which it is derived, the addition of a plurality of helix-promoting amino acids in substituting for amino acids (e.g. of less helical propensity, or charged amino acids) present within the base sequence to result in a synthetic peptide having improved biological activity as compared to a peptide of the base sequence.

Examples of such substitutions include the following, wherein an “h” under the amino acid position indicates addition of a helix-promoting amino acid in place of a charged amino acid (e.g., glutamic acid) or an amino acid of less helical propensity (including, but not limited to amino acids considered to have no helical propensity) in the corresponding amino acid position of SEQ ID NO:3.

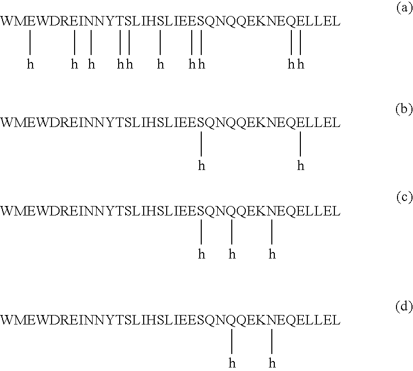

With respect to Examples (b)-(d), the synthetic peptide has two or more substitutions that include at least an “a” position in one heptad and a “d” position in a different (preferably adjacent) heptad surprisingly resulting in improved biological activity, as compared to a peptide of the base sequence of any one or more of SEQ ID NOs: 2, 3, or 4. More preferably, the total number of “a” and “d” positions of the base sequence which are substituted with a helix-promoting amino acid ranges from 2 to 5. In a preferred embodiment, the helix-promoting amino acid is either a leucine or isoleucine, or a combination of leucine and isoleucine, in forming from 1 to 3 additional leucine zipper-like motifs as compared to the base sequence.

In another embodiment, the synthetic peptides according to the present invention have a plurality of additional amino acids as compared to (e.g., substituted for amino acids within) the base sequence consisting of an amino acid sequence of SEQ ID NO:3, which include, but are not limited to, any one of more of the following; wherein a “c” under the amino acid position indicates addition of a charged amino acid in place of an uncharged amino acid in the corresponding amino acid position of SEQ ID NO:3 for forming an ion pair with an appropriately spaced apart oppositely charged amino acid (i.e., charge opposite to the amino acid added); and an “h” under the amino acid position indicates addition of a helix-promoting amino acid in place of a charged amino acid (e.g., glutamic acid) or an amino acid of less helical propensity (including, but not limited to amino acids considered to have no helical propensity) in the corresponding amino acid position of SEQ ID NO:3. 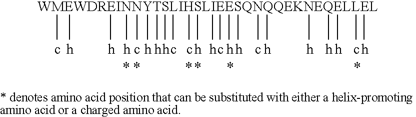
With respect to the “a” and “d” positions of the base sequence which are substituted with a helix-promoting amino acid in producing a synthetic peptide, in a preferred embodiment, the helix-promoting amino acid is either a leucine or isoleucine, or a combination of leucine and isoleucine, in forming from 1 to 3 additional leucine zipper-like motifs as compared to the base sequence of any one or more of SEQ ID NOs: 2, 3, or 4. Illustrations of this embodiment include, but are not limited to, SEQ ID NOs: 97, 98, 99, 100, 101, 102, and 103.

Examples of ion pairs that may be formed in the amino acid positions following substitutions with charged (where “+” represents positively charged, and “−” represents negatively charged) amino acids (indicated by “c”) include, but are not limited to, any one or more of the following.

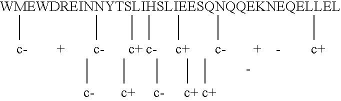

Accordingly, and in a preferred embodiment, a synthetic peptide according to the present invention (when compared to a base sequence of any one of SEQ ID NOs: 2, 3, or 4) has an amino acid sequence having: (a) no less than 2 helix-promoting amino acids, and no more than 14 helix-promoting amino acids, in positions of which corresponding positions of the base sequence lack a helix-promoting amino acid; and (b) no less than 2 charged amino acids, and no more than 10 charged amino acids, in positions of which corresponding positions of the base sequence lack a charged amino acid. An amino acid sequence of SEQ ID NO:104 is representative of a general formula for such synthetic peptides according to the present invention, wherein “Xaa” represents any amino acid (naturally or non-naturally occurring); “Zaa” is used to denote an amino acid that may be either leucine or isoleucine; and “Baa” is used to denote an amino acid that is preferably either leucine, isoleucine, but may be Xaa, except that at least one Baa is either a leucine or isoleucine.

SEQ ID NO:104

XaaXaaXaaEAXaaDRAZaaAEXaaAARZaaEAZaaZaaRABaaXAaE
XaaXaaEKBaaEAAZaaREZaa

Also one or more conservative amino acid substitutions can be made in SEQ ID NO:104, such as a substitution of a charged amino acid with a similarly charged amino acid; with examples including a lysine substituted by an arginine or histidine, an arginine substituted by a lysine or histidine, a glutamic acid substituted by an aspartic acid, or an aspartic acid substituted by a glutamic acid.

B. Helicity

Helicity is a biophysical parameter. The helicity of peptides consisting of a base sequence typically is in a range of from about 9% to about 10%, as assessed by circular dichroism (See Example 1, herein). Synthetic peptides according to the present invention generally have a helicity that is in a range of from about 25% to about 100%, and preferably in a range of from about 48% to about 85%.

C. Size

A synthetic peptide according to the present invention may comprise a sequence of no less than about 15 amino acids and no more than about 60 amino acid residues in length, and preferably no less than 28 amino acids and no more than about 38 amino acids in length. A synthetic peptide according to the present invention is derived from (e.g., comprises a contiguous sequence of at least the contiguous amino acid residues) any one or more of SEQ ID NO:2 or SEQ ID NO:3 or SEQ ID NO:4, or a portion thereof, with inclusion in the synthetic peptide of some amino acids which are different from (substitutions of amino acids in) amino acids in the corresponding position within the base sequence from which the synthetic peptide is derived. The differences in the amino acid sequence (in the synthetic peptide as compared to that of the base sequence from which it is derived), have been found to influence biophysical (e.g., helicity and stability) and biological (e.g., antiviral) parameters described herein in more detail. The synthetic peptide may further comprise one or more conservative substitutions, as compared to the base sequence from which it is derived. As also described herein in more detail, a synthetic peptide according to the present invention may further comprise a macromolecular carrier.

D. Improved Biological Activity

It is an important feature of each of the synthetic peptides according to the present invention to show improved biological activity. The improved biological activity was unexpected for reasons including the following. For classes (a class usually referring to mechanism of action) of antiretroviral agents to date, such as reverse transcriptase inhibitors and protease inhibitors, a simple viral mutation (in just one or more amino acid residues) can result in reduced or a loss of potency of (“resistance” to) a class of antiretrovirals against such viral mutants. For example, a single mutation, particularly in a codon for the connecting loop of the HIV-1 reverse transcriptase fingers subdomain (e.g., at codon 69 or codon 151) is associated with broad cross resistance to all nucleoside reverse transcriptase inhibitors. While non-nucleoside reverse transcriptase inhibitors (NNRTI) may be chemically diverse, a single mutation (at amino acid 103 which is believed to be in the hydrophobic cavity or NNRTI binding site of the reverse transcriptase) results in broad cross-resistance to NNRTIs. Despite the structural diversity of the protease inhibitors (PIs), HIV-1 strains have emerged possessing cross-resistance to all members of this class. More particularly, a limited number of mutations (e.g., combined substitutions at amino acids 10 and 90 by themselves, and also in the presence of other mutations) in the HIV protease results in broad cross-resistance to PIs. Therefore, one of reasonable skill in the art would expect that a single or limited number of mutations in the gp41 amino acid sequence (e.g., in the HR1 region) would confer resistance to the broad class of fusion inhibitor peptides derived from HIV gp41 (e.g., including synthetic peptides according to the present invention). Accordingly, it is an unexpected result that synthetic peptides, derived from the HR2 region and which have been modified in the amino acid sequence according to the present invention, can demonstrate improved biological activity (i.e., increased antiviral potency) against viral mutations which render the virus resistant to peptides derived from the native sequence of HIV gp41. For example, a synthetic peptide according to the present invention comprises unexpected, improved biological activity when, against a virus isolate resistant to peptides derived from the native sequence of HIV-1 gp41 (e.g., a base sequence, or T20 (SEQ ID NO:1)), by demonstrating an IC50 of less than 0.5 μg/ml, preferably less than 0.3 μg/ml, and more preferably less than 0.10 μg/ml. Thus, a synthetic peptide according to the present invention does not consist of any one of SEQ ID NOs: 105, 106, 107, and 108, which lack such antiviral activity against such viral isolate, as shown in Table 2.

Additionally, in a preferred embodiment, a synthetic peptide according to the present invention has both an increase in antiviral activity against a virus resistant to peptides derived from the native sequence of HIV-1 gp41 (e.g., a base sequence from which the synthetic peptide is derived), and improved pharmacokinetic properties as compared to a base sequence from which the synthetic peptide is derived. For example, with respect to base sequences consisting of the amino acid sequences of SEQ ID NO:2 and SEQ ID NO:3, the clearance values (in L/K/hr) are each greater than 0.30, as measured using the methods described herein. For comparison purposes and using the same methods of measurement, a synthetic peptide according to the present invention may have a clearance value that ranges from about 0.005 to about 0.07 (expressed in UK/hr). Thus, preferably, the improved pharmacokinetic properties are illustrated by no less than a 30% reduction in clearance. In another example, with respect to base sequences consisting of the amino acid sequences of SEQ ID NO:2 and SEQ ID NO:3, the biological half-life (also termed herein as “terminal elimination half-life” or “t ½”; expressed in hours (hr) or fraction thereof) are each less than 0.50 hr, as measured using the methods described herein. For comparison purposes and using the same methods of measurement, a synthetic peptide according to the present invention may have a biological half-life that ranges from about 3 hr to greater than 20 hr. Thus, the improved pharmacokinetic properties of a synthetic peptide are illustrated by no less than a 5 fold increase in biological half-life; preferably, no less than a 10 fold increase in biological half-life; and more preferably, no less than a 30 fold increase in biological half-life.

E. Stability

Stability is a biophysical parameter well known in the art of proteins and peptides. There are various methods for determining stability, as known to those skilled in the art. In a preferred embodiment, a synthetic peptide according to the present invention comprises a stability represented by a melting temperature (“Tm”) in the range of from about 25° C. to about 75° C., and more preferably from about 36° C. to about 65° C.

As described herein in more detail, a synthetic peptide may further comprise a component selected from the group consisting of one or more reactive functionalities (e.g., at either the C-terminal end, or N-terminal end, or a combination thereof (both the C-terminal end and N-terminal end)), a pharmaceutically acceptable carrier, a macromolecular carrier, and a combination thereof.

The present invention is illustrated in the following examples, which are not intended to be limiting.

### EXAMPLE 1

In the following examples, various biophysical parameters and biological parameters were assessed. The general methodologies for determining these parameters are as follows.

Peptides consisting of base sequences and synthetic peptides were synthesized on a peptide synthesizer using standard solid-phase synthesis techniques and using standard FMOC peptide chemistry. In this example, the synthetic peptides may further comprise reactive functionalities; i.e., most were blocked at the N-terminus by an acetyl group and/or at the C-terminus by an amide group, or comprised a linker at the N-terminus or C terminus. After cleavage from the resin, the peptides were precipitated, and the precipitate was lyophilized. The peptides were then purified using reverse-phase high performance liquid chromatography; and peptide identity was confirmed with electrospray mass spectrometry.

Helicity was assessed by circular dichroism (“CD”) as follows. Briefly, CD spectra were obtained using a spectrometer equipped with a thermoelectric temperature controller. The spectra was obtained at 25° C. with 0.5 nanometer (nm) steps from 200 to 260 nm, with a 1.5 nm bandwith, and a typical averaging time of 4 seconds/step. After the cell/buffer blank was subtracted, spectra were smoothed using a third-order least-squares polynomial fit with a conservative window size to give random residuals. Raw ellipticity values were converted to mean residue ellipticity using standard methods, and plotted was the wavelength (from 200 to 260 nm) versus [θ]×10-3 (degrees cm²/dmol). Percent helicity values were then calculated using standard methods (usually expressed as percent helicity at 10 μM, 25° C.). Assessment of thermal stability was performed by monitoring the change in CD signal at 222 nm as temperature was raised in 2° C. steps, with 1 minute equilibration times. The stability for each sample (e.g., synthetic peptide), as represented by the Tm value, is the temperature corresponding to the maximum value of the first derivative of the thermal transition.

In determining antiviral activity (e.g., one measure being the ability to inhibit transmission of HIV to a target cell) of the synthetic peptides according to the present invention, used was an in vitro assay which has been shown, by data generated using peptides derived from the HR regions of HIV gp41, to be predictive of antiviral activity observed in vivo. More particularly, antiviral activity observed using an in vitro infectivity assay (“Magi-CCR5 infectivity assay”; see, e.g., U.S. Pat. No. 6,258,782) has been shown to reasonably correlate to antiviral activity observed in vivo for the same HIV gp41 derived peptides (see, e.g., Kilby et al., 1998, Nature Med. 4:1302-1307). These assays score for reduction of infectious virus titer employing the indicator cell lines MAGI or the CCR5 expressing derivative cMAGI. Both cell lines exploit the ability of HIV-1 tat to transactivate the expression of a β-galactosidase reporter gene driven by the HIV-LTR. The β-gal reporter has been modified to localize in the nucleus and can be detected with the X-gal substrate as intense nuclear staining within a few days of infection. The number of stained nuclei can thus be interpreted as equal to the number of infectious virions in the challenge inoculum if there is only one round of infection prior to staining. Infected cells are enumerated using a CCD-imager and both primary and laboratory adapted isolates show a linear relationship between virus input and the number of infected cells visualized by the imager. In the MAGI and cMAGI assays, a 50% reduction in infectious titer (Vn/Vo=0.5) is significant, and provides the primary cutoff value for assessing antiviral activity (“IC50” is defined as the concentration of active ingredient resulting in a 50% reduction in infectious virus titer). Peptides tested for antiviral activity were diluted into various concentrations, and tested in duplicate or triplicate against an HIV inoculum adjusted to yield approximately 1500-2000 infected cells/well of a 48 well microtiter plate. The peptide (in the respective dilution) was added to the cMAGI or MAGI cells, followed by the virus inocula; and 24 hours later, an inhibitor of infection and cell-cell fusion (e.g., T20) was added to prevent secondary rounds of HIV infection and cell-cell virus spread. The cells were cultured for 2 more days, and then fixed and stained with the X-gal substrate to detect HIV-infected cells. The number of infected cells for each control and peptide dilution was determined with the CCD-imager, and then the IC50 was calculated (expressed in μg/ml).

Viruses resistant to the antiviral activity of a peptide consisting of a base sequence can be produced using standard laboratory methods. Basically, after calculating the IC50 and IC90, cells were mixed with virus and the peptide (e.g., at a concentration close to the IC90) in culture (including when the cells are split thereafter). The cultures are maintained and monitored until syncytia are present. Virus harvested from this first round of culture is used to infect cells in a second round of culture, with the peptide present in a higher concentration (2 to 4 times) than that used in the first round of culture. The second round of culture is maintained and monitored for presence of virus resistant to the antiviral activity of the peptide. Subsequent rounds of culture may be needed to finally generate a viral isolate resistant to the antiviral activity of the peptide (at a pre-determined level of the IC50 of the peptide against such isolate).

For determining pharmacokinetic properties, a synthetic peptide or a base sequence from which a synthetic peptide is derived, was dosed intravenously in cynomolgus monkeys (Macaca fasicularis) (other animal models may be used for determining pharmacokinetic properties, as known in the art). At various times post-dose, blood samples were drawn and plasma isolated by centrifugation. Plasma samples were stored frozen until analysis by LC-MS (liquid chromatography/mass spectrometry) in the electrospray, positive-ion mode. A synthetic peptide or base sequence was eluted from a C18 HPLC column with a gradient of acetonitrile in a buffer of 10 mM ammonium acetate, pH 6.8. At the time of analysis, plasma samples were deproteinated with either two or three volumes of acetonitrile containing 0.5% formic acid. Duplicate calibration standards in cynomolgus plasma samples were prepared at the same time as the samples and analyzed before and after the samples containing either synthetic peptide or base sequence. Pharmacokinetic properties were calculated from the plasma concentration-time data using either mono-exponential or bi-exponential mathematical models. Models were derived by non-linear least squares optimization. A 1/C² weighting of concentrations was used. The following equations were used to calculate area-under the plasma concentration vs. time curve (AUC), total body clearance (CI), and terminal elimination half-life (t1½).
AUC=A/−a+B/−b
Where A and B are intercepts and a and b are the rate constants of the exponential equations describing the distribution and elimination phases, respectively. When mono-exponential models were used, the “A” and “a” properties were eliminated.
Cl=Dose/AUC (expressed in L/K/hr)
t½=−0.6903/b (expressed in hr)

### EXAMPLE 2

In one embodiment according to the present invention, a synthetic peptide was synthesized except that, as compared to the base sequence from which it's amino acid sequence is derived, added was a plurality of amino acids comprising one or more helix-promoting amino acids. As exemplified by a synthetic peptide having an amino acid sequence of SEQ ID NO:5, the synthetic peptide according to the present invention was synthesized to comprise a plurality of helix-promoting amino acid substitutions in relation to base sequence consisting of SEQ ID NO:4. With reference to Table 1, synthetic peptide according to the present invention was compared to peptides having a base sequence of either SEQ ID NO:2 or SEQ ID NO:4 for biophysical parameters and biological parameters, as determined using the methodology described in Example 1 herein. In determining biological activity as assessed by antiviral activity, utilized were virus mutants which are resistant to the antiviral activity of peptides having the base amino acid sequence of SEQ ID NO:2 or SEQ ID NO:4 (the resistant viral isolate being designated as “RY” in Table 1 and subsequent Tables).

With reference to Table 1, as compared to a peptide of the native sequence of HR2 (e.g., any one of base sequences SEQ ID NO:2 or SEQ ID NO:4) from which it was derived, a synthetic peptide according to the present invention: (a) demonstrates an increase in helicity (e.g., an increase in a range of from about 3 fold to about 5 fold or greater); and (b) demonstrates a significant increase in antiviral activity, an unexpected, improved biological activity, against virus resistant to peptides having any one of the base sequences (e.g., SEQ ID NOs:2 or 4) (e.g., virus isolate HIV RY).

TABLE 1
Biophysical and Biological (antiviral activity) Parameters

| | | | Antiviral | Antiviral |
|---|---|---|---|---|
| SEQ ID | Helicity | | Activity | Activity |
| NO: | (%) | Tm (° C.) | HIV-IIIB IC50 | HIV-RY IC50 |
| 2 | 9 | <10 | <0.10 | >4.0 |
| 4 | 10 | <10 | <0.10 | >4.0 |
| 5 | 51 | >20 | <0.10 | <0.30 |
| 82 | 11 | >10 | <0.10 | <0.20 |
| 84 | 18 | >15 | <0.10 | <0.20 |
| 85 | 91 | 45 | <0.10 | <0.10 |
| 86 | 66 | 30 | <0.10 | <0.30 |
| 87 | 11 | | <0.10 | <0.10 |

### EXAMPLE 3

In another embodiment, produced was a synthetic peptide comprising an addition of amino acids comprising helix-promoting amino acids, and charged amino acids (in forming a plurality of ion pairs), in place of amino acids present within any one or more of base sequences SEQ ID NOs: 2-4. For purposes of illustration, synthetic peptides, exemplified by an amino acid sequence having any one of SEQ ID NOs:6-81, and 83-95, and 97-98 were produced and assessed using the methods outlined in Example 1 herein. With reference to Table 2, these synthetic peptides were compared to a peptide derived from that native sequence of the HR2 region (e.g., base sequence of SEQ ID NO:4), and against peptides having substitutions solely consisting of charged amino acids placed in an i, i+4 arrangements (e.g., without addition of a plurality of helix-promoting amino acids) such as SEQ ID NOs:105-108, for biophysical parameters and biological parameters using methods as previously described in more detail in Example 1 herein.

TABLE 2
Biophysical and Biological (antiviral activity) Parameters

| | | | Antiviral | Antiviral |
|---|---|---|---|---|
| SEQ ID | Helicity | | Activity | Activity |
| NO: | (%) | Tm (° C.) | HIV-IIIB IC50 | HIV-RY IC50 |
| 4 | 10 | 10 | <0.10 | >4.0 |
| 6 | 72 | 38 | <0.10 | <0.10 |
| 7 | 45 | 25 | <0.10 | <0.10 |
| 8 | 74 | 48 | <0.10 | <0.10 |
| 9 | 75 | 56 | <0.10 | <0.10 |
| 10 | 67 | 48 | <0.10 | <0.10 |
| 11 | 65 | 42 | <0.10 | <0.10 |
| 12 | 73 | 45 | <0.10 | <0.10 |
| 13 | 83 | 57 | <0.10 | <0.10 |
| 14 | 90 | 62 | <0.20 | <0.30 |
| 15 | 87 | 59 | <0.20 | <0.30 |
| 16 | 66 | 41 | <0.10 | <0.10 |
| 17 | 59 | 43 | <0.10 | <0.10 |
| 18 | 65 | 47 | <0.10 | <0.10 |
| 19 | 59 | 42 | <0.10 | <0.10 |
| 20 | 60 | 44 | <0.10 | <0.10 |
| 21 | 64 | 45 | <0.10 | <0.10 |
| 22 | 67 | 46 | <0.10 | <0.10 |
| 23 | 56 | 41 | <0.10 | <0.10 |
| 24 | 71 | 40 | <0.10 | <0.10 |
| 25 | 89 | 42 | <0.10 | <0.10 |
| 26 | 82 | 38 | <0.10 | <0.10 |
| 27 | 88 | 59 | <0.10 | <0.10 |
| 28 | 68 | 39 | <0.10 | <0.10 |
| 29 | 82 | 42 | <0.10 | <0.10 |
| 30 | 78 | 43 | <0.10 | <0.10 |
| 31 | 54 | 29 | <0.10 | <0.10 |
| 32 | 61 | 31 | <0.10 | <0.10 |
| 33 | 63 | 34 | <0.10 | <0.10 |
| 34 | 69 | 36 | <0.10 | <0.10 |
| 35 | 6 | <10 | <0.10 | <0.10 |
| 36 | Agg | Agg | <0.10 | <0.10 |
| 37 | 80 | 43 | <0.10 | <0.20 |
| 38 | 65 | 49 | <0.10 | <0.20 |
| 39 | 78 | 44 | <0.10 | <0.10 |
| 40 | 68 | 42 | <0.10 | <0.10 |
| 41 | 96 | 65 | <0.10 | <0.10 |
| 42 | 97 | 64 | <0.10 | <0.10 |
| 43 | 92 | 65 | <0.10 | <0.10 |
| 44 | 55 | 37 | <0.10 | <0.10 |
| 45 | 61 | 39 | <0.10 | <0.20 |
| 46 | 70 | 41 | <0.10 | <0.10 |
| 47 | 73 | 42 | <0.10 | <0.10 |
| 48 | 65 | 39 | <0.10 | <0.10 |
| 49 | 63 | 37 | <0.10 | <0.50 |
| 50 | 90 | 59 | <0.10 | <0.10 |
| 51 | 97 | 65 | <0.10 | <0.10 |
| 52 | >99 | 72 | <0.10 | <0.20 |
| 53 | 94 | 59 | <0.10 | <0.10 |
| 54 | 95 | 75 | <0.10 | <0.10 |
| 55 | 57 | 25 | <0.10 | <0.10 |
| 56 | 57 | 28 | <0.10 | <0.10 |
| 57 | 73 | 39 | <0.10 | <0.20 |
| 58 | 88 | 41 | <0.10 | <0.10 |
| 59 | 89 | 46 | <0.10 | <0.10 |
| 60 | 78 | 46 | <0.10 | <0.10 |
| 61 | 41 | 25 | <0.10 | <0.10 |
| 62 | | | <0.10 | <0.10 |
| 63 | 65 | 38 | <0.10 | <0.10 |
| 64 | 91 | 41 | <0.10 | <0.10 |
| 65 | 38 | | <0.10 | <0.20 |
| 66 | 99 | 57 | <0.10 | <0.20 |
| 67 | 95 | 43 | <0.10 | <0.10 |
| 68 | 73 | | ≦0.10 | <0.20 |
| 69 | 77 | | <0.10 | <0.10 |
| 70 | 58 | 36 | <0.10 | <0.20 |
| 71 | 84 | | <0.10 | <0.10 |
| 72 | | | <0.10 | <0.10 |
| 73 | | | <0.10 | <0.20 |
| 74 | | | ≦0.10 | <0.20 |
| 75 | | | <0.10 | <0.10 |
| 76 | | | <0.10 | <0.10 |
| 77 | 67 | 40 | <0.10 | <0.10 |
| 78 | | | <0.10 | <0.10 |
| 79 | 72 | 38 | <0.10 | <0.10 |
| 80 | 80 | 61 | ≦0.10 | <0.20 |
| 81 | 91 | 46 | <0.10 | <0.10 |
| 83 | 34 | 11 | <0.10 | <0.10 |
| 88 | | | <0.10 | <0.10 |
| 89 | 89 | 44 | <0.10 | <0.10 |
| 90 | 80 | 44 | <0.10 | <0.10 |
| 91 | 95 | 69 | <0.10 | <0.10 |
| 92 | 97 | 73 | <0.10 | <0.10 |
| 93 | 93 | 70 | <0.10 | <0.10 |
| 94 | 96 | 83 | <0.10 | <0.20 |
| 95 | 90 | 53 | <0.10 | <0.10 |
| 97 | 61 | 62 | <0.10 | <0.10 |
| 98 | 77 | 75 | <0.10 | <0.10 |
| 105 | 17 | <10 | <0.10 | >2.0 |
| 106 | 37 | <10 | <0.10 | >0.50 |
| 107 | 47 | 23 | <0.10 | >1.0 |
| 108 | | | <0.10 | >10.0 |

Agg—aggregated

With reference to Table 2, as compared to a peptide of the native sequence of JR2 (e.g., any one of base sequences SEQ ID NOs:2-4) from which it was derived, a synthetic peptide according to the present invention demonstrates a significant increase in antiviral activity, an unexpected, improved biological activity, against virus resistant to peptides having any one of the base sequences (e.g., SEQ ID NOs:2 or 4) (e.g., virus isolate HIV RY). Additionally, a synthetic peptide may further demonstrates an increase in helicity (e.g., in a range of from about 3 fold to about 5 fold or greater), as compared to any one of the base sequences (e.g., SEQ ID NOs:2 or 4). However, note that this unexpected, improved biological activity was not observed for peptides having an amino acid sequence of any one of SEQ ID NOs:105-108, which consist of substitutions within a base sequence consisting of charged amino acids (i.e., without addition of any helix-promoting amino acids).

With reference to Table 2 and in another preferred embodiment, as compared to a peptide of the native sequence of HR2 (e.g., any one of base sequences SEQ ID NOs:2-4) from which it was derived, a synthetic peptide according to the present invention additionally (e.g., in addition to demonstrating an increase in helicity and unexpected, improved biological activity) and preferentially demonstrates a stability as, for example, measured by a Tm in the range of from about 25° C. to about 75° C., and more preferably from about 36° C. to about 65° C.

Tables 1 and 2 demonstrate the unexpected, improved biological activity of a synthetic peptide according to the present invention, as assessed by (a) determining the antiviral activity of the synthetic peptide against an HIV strain that demonstrates resistance to the activity of a base sequence from which the synthetic peptide is derived; and (b) demonstrating the synthetic peptide has antiviral activity, as measured by an IC50 of less than 0.30 μg/ml, and more preferably an IC50 of less than 0.10 pg/ml, against the HIV strain that demonstrates resistance to the activity of a peptide consisting of abase sequence from which the synthetic peptide is derived (e.g., any one of SEQ ID NOs: 2-4). In another demonstration of such unexpected biological activity, synthetic peptide according to the present invention was used in attempts to generate resistant virus in vitro. Thus, for example, a demonstration that it is more difficult to generate resistant virus to a synthetic peptide is evidence that such synthetic peptide has unexpected, improved biological activity, as compared to a peptide of native sequence from the HR2 region of gp41 and/or a base sequence.

Using the methods outlined in Example 1, synthetic peptides having the amino acid sequences of SEQ ID NO:9, SEQ ID NO:10, & SEQ ID NO:97 were compared to a peptide having the base sequence of SEQ ID NO:2 in experiments designed to generate resistant HIV. In one set of experiments, an in vitro culture of HIV-infected cells was passaged in the presence of either the individual synthetic peptide or the base sequence in efforts to reach an endpoint of generation of an isolate of HIV which was resistant at a concentration of 10 μg/ml to 20 μg/ml of the peptide with which the HIV-infected cells were incubated. Thus, typically, starting with the synthetic peptide or base sequence at a concentration between it's IC50 and IC90, the HIV-infected cells were cultured in vitro, and split every 2 to 3 days adding the synthetic peptide or base sequence during the split to maintain the HIV-infected cells in the presence of a constant and consistent amount of synthetic peptide or base sequence. Upon generating a resistant isolate (as measured by cytopathic effectsyncytia formation; considered as one passage) at that low level of concentration of synthetic peptide or base sequence, cells were infected with the resistant isolate generated from that passage, and the cells were then cultured in the presence of a higher concentration (e.g., 2 to 3 times the concentration used in the previous passage) of synthetic peptide or base sequence until a resistant HIV isolate was generated. This procedure was repeated until achieved is the endpoint. Determined is the number of passages, and number of days (number of days in each successful passage (where a viable virus was generated) then totaled together for all successful passages), in culture required to reach the endpoint. Results were averaged for each base sequence or synthetic peptide illustrated. As shown in Table 3, unexpectedly, to generate resistant HIV isolates representative of the endpoint, significantly more passages (Table 3, “Passage #”) and days in passages (Table 3, “Days #”) in the presence of a synthetic peptide of the present invention are required, if achieved at all, as compared to the base sequence. The results in Table 3 are another indication of the unexpected, improved biological activity demonstrated by a synthetic peptide, as compared to a base sequence from which the synthetic peptide is derived.

TABLE 3
in vitro resistance generation

| SEQ ID NO: | Average # Passages | Average # Days |
|---|---|---|
| SEQ ID NO: 2 | 15 | 115 |
| SEQ ID NO: 9 | 19 | 188 |
| SEQ ID NO: 10 | >20 NA | >200 NA |
| SEQ ID NO: 97 | >5 NA | 74 NA |

“NA” means endpoint not achieved.

### EXAMPLE 4

Illustrated in this example is the improved pharmacokinetic properties of a synthetic peptide according to the present invention as compared to a base sequence from which the synthetic peptide is derived. Using methods for assessing pharmacokinetic properties as previously described in more detail in Example 1, Table 4 illustrates pharmacokinetic properties of a representation of synthetic peptides as compared to the pharmacokinetic properties of a base sequence consisting of either SEQ ID NO:2 or SEQ ID NO:3.

TABLE 4

| | | Half-life |
|---|---|---|
| SEQ ID NO: | Clearance (L/K/hr) | (t ½; hr) |
| 2 | >0.4 | <0.5 |
| 3 | >0.3 | <0.5 |
| 10 | <0.05 | >5.0 |
| 11 | <0.05 | >15.0 |
| 49 | <0.05 | >7.5 |
| 50 | <0.10 | >3.0 |
| 51 | <0.05 | >15.0 |
| 52 | <0.05 | >10.0 |
| 53 | <0.05 | >5.0 |
| 54 | <0.01 | >20.0 |
| 76 | <0.05 | >7.5 |
| 78 | <0.05 | >7.5 |
| 79 | <0.10 | >3.0 |
| 80 | <0.05 | >5.0 |
| 91 | <0.05 | >15.0 |
| 92 | <0.01 | >10.0 |
| 93 | <0.05 | >7.5 |
| 94 | <0.01 | >7.5 |
| 95 | <0.10 | >10.0 |
| 97 | <0.01 | >15.0 |
| 98 | <0.05 | >20.0 |

As illustrated in Table 4, a synthetic peptide, as compared to a base sequence from which it was derived, exhibited a marked improvement in pharmacokinetic properties as observed in one or more pharmacokinetic properties (e.g., in either or both of clearance and t½). Preferably, the improved pharmacokinetic properties are illustrated by no less than a 30% reduction in clearance. Preferably, the improved pharmacokinetic properties of a synthetic peptide are illustrated by no less than a 5 fold increase in biological half-life; preferably, no less than a 10 fold increase in biological half-life; and more preferably, no less than a 30 fold increase in biological half-life.

For formulating an HIV fusion inhibitor into a pharmaceutically acceptable carrier in producing a pharmaceutical composition, stability in aqueous solution may be an important parameter, particularly if the pharmaceutical composition is to be administered parenterally. It is noted that a synthetic peptide according to the present invention demonstrates improvement in stability in aqueous solutions at physiological pH. For example, a peptide having an amino acid sequence of SEQ ID NO:1 was compared with a synthetic peptide having an amino acid sequence of SEQ ID NO:11, and a synthetic peptide having an amino acid sequence of SEQ ID NO:97 for stability in solution. Each was individually tested for stability in aqueous solution by adding the peptide at a concentration of 10 mg/ml to phosphate-buffered saline (PBS), and by measuring (e.g., by HPLC) at different time points over a period of 1 week (168 hours) the amount of peptide remaining in solution at a range of about pH 7.3 to about pH 7.5 at 37° C. A solution containing a peptide having the amino acid sequence of SEQ ID NO:1 becomes unstable after just several hours (minimal to no peptide detected in solution). In contrast, 90% or more of a synthetic peptide having an amino acid sequence of SEQ ID NO:97 remains detectable in solution at a time point of 1 week, and more than 70% of a synthetic peptide having the amino acid sequence of SEQ ID NO:11 remains detectable in solution at a time point of 1 week; both synthetic peptides demonstrating good to excellent stability in aqueous solution.

### EXAMPLE 5

The present invention provides for synthetic peptides according to the present invention, which possess antiviral activity as evidenced by their ability to inhibit transmission of HIV (including, unexpectedly, isolates resistant to a base sequence from which synthetic peptide was derived) to a target cell (e.g., see Tables 1 & 2). Additionally, provided are uses of synthetic peptide according to the present invention. For example, a synthetic peptide according to the present invention may be used as an active therapeutic substance in therapy of HIV infection. Also, a synthetic peptide according to the present invention may be used for the manufacture of a medicament for a therapeutic application comprising treatment of HIV. Additionally, a synthetic peptide according to the present invention may be used for treatment of HIV, including a therapeutic application thereof (e.g., reducing the viral load of HIV, and/or increasing the CD4+ cell population, in a treated individual). In one embodiment, a method of treating an HIV-infected individual comprises administering to the individual an amount of a synthetic peptide (including a composition/medicament in which the synthetic peptide is an active therapeutic substance) effective to treat the individual or to achieve the desired therapeutic application. With respect to the latter, a baseline value (from measuring the parameter of viral load and/or CD4+ cell count) is obtained from a clinical sample prior to treatment with the synthetic peptide. One or more clinical samples are obtained subsequent to the initiation of treatment with synthetic peptide, and from such sample(s) is measured the parameter (“test value”). The baseline value and test value are compared to determine if the desired therapeutic application was achieved from treatment with synthetic peptide (e.g., a difference between the test value and a baseline value may be an indication that the desired therapeutic application was achieved).

In another embodiment, provided is a method for inhibiting transmission of HIV to a target cell comprising adding to the virus and the cell an amount of synthetic peptide according to the present invention effective to inhibit infection of the cell by HIV. In another embodiment, provided is a method for inhibition of transmission of HIV to a cell, comprising contacting the virus in the presence of a cell with an amount of synthetic peptide according to the present invention effective to inhibit infection of the cell by HIV. Additionally, provided is a method for inhibiting HIV fusion (e.g., a process by which HIV gp41 mediates fusion between the viral membrane and cell membrane during infection by HIV of a target cell), comprising contacting the virus in the presence of a cell with an amount of synthetic peptide according to the present invention effective to inhibit HIV fusion. These methods may be used to treat HIV-infected individuals (therapeutically) or to treat individuals newly exposed to or at high risk of exposure (e.g., through drug usage or high risk sexual behavior) to HIV (prophylactically). Thus, for example, in the case of an HIV-1 infected individual, an effective amount would be a dose sufficient (by itself and/or in conjunction with a regimen of doses) to reduce HIV viral load in the individual being treated. As known to those skilled in the art, there are several standard methods for measuring HIV viral load which include, but are not limited to, by quantitative cultures of peripheral blood mononuclear cells, by plasma HIV RNA measurements, and by measuring the viral nucleic acids by a quantitative method involving nucleic acid amplification using standard methods known in the art. Methods for determining CD4⁺ cell levels (a “CD4⁺ cell count”) are standard in the art. Such methods include, but are not limited to, flow cytometry, immunoassay, magnetic separation followed by cell counting, immunocytochemical, and immunostaining. Standards for HIV viral load and CD4⁺ cell counts which are indicative of various stages of HIV infection and AIDS are well known in the art. One source for such standards is the Centers for Disease Control.

The synthetic peptides of the invention can be administered in a single administration, intermittently, periodically, or continuously, as can be determined by a medical practitioner using methods such as monitoring viral load and/or blood levels of synthetic peptide. Depending on the formulation containing synthetic peptide, and whether the synthetic peptide further comprises a macromolecular carrier, the synthetic peptides according to the present invention may be administered once or multiple times daily, or periodically during a week period, or periodically during a month period. Further, the synthetic peptides according to the present invention may show synergistic results or added therapeutic benefit of inhibiting transmission of HIV to a target cell, when used as a component in a combination or a therapeutic regimen (e.g., when used simultaneously, or in a cycling on with one drug and cycling off with another) containing one or more additional antiviral drugs used for treatment of HIV including, but not limited to, HIV entry inhibitors (e.g., other HIV fusion inhibitors (T20, T1249, and the like), CCR5 inhibitors, retrocyclins, etc.), HIV integrase inhibitors, reverse transcriptase inhibitors (e.g., nucleoside or nonnucleoside), protease inhibitors, viral-specific transcription inhibitors, viral processing inhibitors, HIV maturation inhibitors, inhibitors of uridine phosphorylating enzyme, HIV vaccines, and the like, as well known in the art.

For example, in one preferred embodiment, combinations of antiviral agents may be used which include one or more synthetic peptides according to the present invention, thus increasing the efficacy of the therapy, and lessening the ability of the virus to become resistant to the antiviral drugs. Combinations may be prepared from effective amounts of antiviral agents (useful in treating of HIV infection) currently approved or approved in the future, which include, but are not limited to, reverse transcriptase inhibitor, including but not limited to, abacavir, AZT (zidovudine), ddC (zalcitabine), nevirapine, ddl (didanosine), FTC (emtricitabine), (+) and (−) FTC, reverset, 3TC (lamivudine), GS 840, GW-1592, GW-8248, GW-5634, HBY097, delaviridine, efavirenz, d4T (stavudine), FLT, TMC125, adefovir, tenofovir, and alovudine; protease inhibitor, including but not limited to, amprenivir, CGP-73547, CGP-61755, DMP-450, indinavir, nelfinavir, PNU-140690, ritonavir, saquinavir, telinavir, tipranovir, atazanavir, lopinavir, darunivir; viral entry inhibitor, including but not limited to, fusion inhibitor (enfuvirtide, T-1249, other fusion inhibitor peptides, and small molecules), chemokine receptor antagonist (e.g., CCR5 antagonist, such as ONO-4128, GW-873140, AMD-887, CMPD-167; CXCR4 antagonist, such as AMD-070), an agent which affects viral binding interactions (e.g., affects gp120 and CD4 receptor interactions, such as BMS806, BMS-488043; and/or PRO 542, PRO140; anti-CD4 antibody; or lipid and/or cholesterol interactions, such as procaine hydrochloride (SP-01 and SP-01A)); integrase inhibitor, including but not limited to, L-870, and 810; RNAseH inhibitor; inhibitor of rev or REV; inhibitor of vif (e.g., vif-derived proline-enriched peptide, HIV-1 protease N-terminal-derived peptide); viral processing inhibitor, including but not limited to betulin, and dihydrobetulin derivatives (e.g., PA457); and immunomodulator, including but not limited to, AS-101, granulocyte macrophage colony stimulating factor, IL-2, valproic acid, and thymopentin. As appreciated by one skilled in the art of treatment of HIV infection and/or AIDS, a combination drug treatment may comprise two or more therapeutic agents having the same mechanism of action (viral protein or process as a target), or may comprise two or more therapeutic agents having a different mechanism of action. Effective dosages of these illustrative antiviral agents, which may be used in combinations with synthetic peptide according to the present invention, are known in the art. Such combinations may include a number of antiviral agents that can be administered by one or more routes, sequentially or simultaneously, depending on the route of administration and desired pharmacological effect, as is apparent to one skilled in the art.

Effective dosages of the synthetic peptides of the invention to be administered may be determined through procedures well known to those in the art; e.g., by determining potency, biological half-life, bioavailability, and toxicity. In a preferred embodiment, an effective synthetic peptide dosage range is determined by one skilled in the art using data from routine in vitro and in vivo studies well know to those skilled in the art. For example, in vitro infectivity assays of antiviral activity, such as described herein, enables one skilled in the art to determine the mean inhibitory concentration (IC) of the synthetic peptide necessary to block some amount of viral infectivity (e.g., 50% inhibition, IC₅₀; or 90% inhibition, IC₉₀). Appropriate doses can then be selected by one skilled in the art using pharmacokinetic data from one or more standard animal models, so that a minimum plasma concentration (C[min]) of the peptide is obtained which is equal to or exceeds a predetermined IC value. While dosage ranges typically depend on the route of administration chosen and the formulation of the dosage, an exemplary dosage range of the synthetic peptide according to the present invention may range from no less than 0.1 μg/kg body weight and no more than 10 mg/kg body weight; preferably a dosage range of from about 0.1-100 μg/kg body weight; and more preferably, a dosage of between from about 10 mg to about 250 mg of synthetic peptide. For example, if synthetic peptide according to the present invention further comprises a macromolecular carrier that causes synthetic peptide to remain active in the blood longer than synthetic peptide alone (i.e., in achieving a longer circulating plasma concentration), the amount of synthetic peptide in the dosage may be reduced as compared to the amount of synthetic peptide in a formulation not containing macromolecular carrier, and/or administered less frequently than a formulation not containing macromolecular carrier.

The compositions, including a medicament, of the present invention (e.g., synthetic peptide, preferably with one or more of a pharmaceutically acceptable carrier and a macromolecular carrier) may be administered to an individual by any means that enables the active agent to reach the target cells (cells that can be infected by HIV). Thus, the compositions of this invention may be administered by any suitable technique, including oral, parenteral (e.g., intramuscular, intraperitoneal, intravenous, or subcutaneous injection or infusion, intradermal, or implant), nasal, pulmonary, vaginal, rectal, sublingual, or topical routes of administration, and can be formulated in dosage forms appropriate for each route of administration. The specific route of administration will depend, e.g., on the medical history of the individual, including any perceived or anticipated side effects from such administration, and the formulation of synthetic peptide being administered (e.g., the nature of the pharmaceutically acceptable carrier and/or macromolecular carrier of which synthetic peptide may further comprise). Most preferably, the administration is by injection (using, e.g., intravenous or subcutaneous means), but could also be by continuous infusion (using, e.g., slow-release devices or minipumps such as osmotic pumps, and the like). A formulation may comprise synthetic peptide according to the present invention which further comprises one or more of a pharmaceutically acceptable carrier and a macromolecular carrier; and may further depend on the site of delivery, the method of administration, the scheduling of administration, and other factors known to medical practitioners. A preferable formulation is one in which synthetic peptide according to the present invention is combined with or further comprises one or more of an agent, drug, reactive functionality, macromolecular carrier, or pharmaceutically acceptable carrier that inhibits or delays or retards the metabolism/degradation of synthetic peptide, particularly after it is administered to an individual. By way of example, injectable formulations, slow-release formulation, and oral formulations in which synthetic peptide of the invention is protected from hydrolysis by enzymes (e.g., digestive enzymes before absorption, proteolytic enzymes present in the blood, and the like) are embraced herein. Additionally, a formulation may comprise nucleotide sequences encoding synthetic peptide according to the present invention, as described herein in more detail, which upon administration, is expressed in cells of interest using techniques and expression vectors well known in the art.

### EXAMPLE 6

It is apparent to one skilled in the art, that based on the respective amino acid sequences of the synthetic peptides according to the present invention, that polynucleotides (nucleic acid molecules) encoding such synthetic peptides may be synthesized or constructed, and that such synthetic peptides may be produced by recombinant DNA technology as a means of manufacture and/or (for example, in vivo production by introducing such polynucleotides in vivo as a means of gene or cell therapy) for a method of inhibiting transmission of HIV to a target cell. It is apparent to one skilled in the art that more than one polynucleotide sequence can encode a synthetic peptide according to the present invention, and that such polynucleotides may be synthesized on the basis of triplet codons known to encode the amino acids of the amino acid sequence of the synthetic peptide, third base degeneracy, and selection of triplet codon usage preferred by the host cell (e.g., prokaryotic or eukaryotic, species, etc,) in which expression is desired,

For purposes of illustration only, and not limitation, provided as SEQ ID NO:109 is a polynucleotide encoding SEQ ID NO:2, a base sequence, from which, as apparent to one skilled in the art, codon usage will generally apply to polynucleotides encoding synthetic peptides of the present invention. Thus, for example, using SEQ ID NO:109 in relation to SEQ ID NO:2, one skilled in the art could readily construct a polynucleotide encoding SEQ ID NO:5 (see, e.g., SEQ ID NO:110 as an illustrative example). Likewise, and as another example, from this information one skilled in the art could readily construct a polynucleotide encoding SEQ ID NO:11 (see, e.g., SEQ ID NO:111 as an illustrative example). However, it is understood that different codons can be substituted which code for the same amino acid(s) as the original codons. Further, as apparent to one skilled in the art, codon usage may vary slightly between codon usage preferred for bacterial expression, or codon usage preferred for expression in mammalian expression systems. In a preferred embodiment, a polynucleotide encoding a synthetic peptide according to the present invention comprises a nucleic acid molecule encoding a synthetic peptide selected from the group consisting of SEQ ID NOs:5-104, or an amino acid sequence having at least 95% identity (and more preferably, at least 90% identity) with any one or more of SEQ ID NOs:5-104 and differing from a base sequence of any one of SEQ ID NOs:2, 3, and 4 by (i) an addition of a plurality of helix-promoting amino acids as compared to corresponding amino acid positions within the base sequence from which the synthetic peptide is derived; or an addition of a plurality of helix-promoting amino acids as compared to corresponding amino acid positions within the base sequence from which the synthetic peptide is derived, and an addition of a plurality of charged amino acids as compared to the positions corresponding to amino acid positions within the base sequence from which it is derived, and (ii) unexpected, improved biological activity.

In one embodiment, provided is a prokaryotic expression vector containing a polynucleotide encoding a synthetic peptide according to the present invention, and its use for the recombinant production of synthetic peptide. In one example, the polynucleotide may be positioned in a prokaryotic expression vector so that when synthetic peptide is produced in bacterial host cells, it is produced as a fusion protein with sequences which assist in purification of the synthetic peptide. For example, there are sequences known to those skilled in the art which, as part of a fusion protein with a peptide desired to be expressed, facilitates production in inclusion bodies found in the cytoplasm of the prokaryotic cell used for expression and/or assists in purification of fusion proteins containing such sequence. Inclusion bodies may be separated from other prokaryotic cellular components by methods known in the art to include denaturing agents, and fractionation (e.g., centrifugation, column chromatography, and the like). In another example, there are commercially available vectors into which is inserted a desired nucleic acid sequence of interest to be expressed as a protein or peptide such that upon expression, the gene product also contains a plurality of terminal histidine residues (“His tags”) that can be utilized in the purification of the gene product using methods standard in the art.

It is apparent to one skilled in the art that a nucleic acid sequence encoding a synthetic peptide according to the present invention can be inserted into, and become part of a, nucleic acid molecule comprising a plasmid or vectors other than plasmids, and other expression systems can be used including, but not limited to, bacteria transformed with a bacteriophage vector, or cosmid DNA; yeast containing yeast vectors; fungi containing fungal vectors; insect cell lines infected with virus (e. g. baculovirus); and mammalian cell lines having introduced therein (e.g., transfected with) plasmid or viral expression vectors, or infected with recombinant virus (e.g. vaccinia virus, adenovirus, adeno-associated virus, retrovirus, etc.). Successful expression of the synthetic peptide requires that either the recombinant nucleic acid molecule comprising the encoding sequence of the synthetic peptide, or the vector itself, contain the necessary control elements for transcription and translation which is compatible with, and recognized by the particular host system used for expression. Using methods known in the art of molecular biology, including methods described above, various promoters and enhancers can be incorporated into the vector or the recombinant nucleic acid molecule comprising the encoding sequence to increase the expression of the synthetic peptide, provided that the increased expression of the synthetic peptide is compatible with (for example, non-toxic to) the particular host cell system used. As apparent to one skilled in the art, the selection of the promoter will depend on the expression system used. Promoters vary in strength, i.e., ability to facilitate transcription. Generally, for the purpose of expressing a cloned gene, it is desirable to use a strong promoter in order to obtain a high level of transcription of the gene and expression into gene product. For example, bacterial, phage, or plasmid promoters known in the art from which a high level of transcription has been observed in a host cell system comprising E. coli include the lac promoter, trp promoter, recA promoter, ribosomal RNA promoter, the P.sub.R and P.sub.L promoters, lacUV5, ompF, bla, Ipp, and the like, may be used to provide transcription of the inserted nucleotide sequence encoding the synthetic peptide. Commonly used mammalian promoters in expression vectors for mammalian expression systems are the promoters from mammalian viral genes. Examples include the SV40 early promoter, mouse mammary tumor virus LTR promoter, adenovirus major late promoter, herpes simplex virus promoter, and the CMV promoter.

In the case where expression of the synthetic peptide may be lethal or detrimental to the host cells, the host cell strain/line and expression vectors may be chosen such that the action of the promoter is inhibited until specifically induced. For example, in certain operons the addition of specific inducers is necessary for efficient transcription of the inserted DNA (e.g., the lac operon is induced by the addition of lactose or isopropylthio-beta-D-galactoside (“IPTG”); trp operon is induced when tryptophan is absent in the growth media; and tetracycline can be use in mammalian expression vectors having a tet sensitive promoter). Thus, expression of the synthetic peptide may be controlled by culturing transformed or transfected cells under conditions such that the promoter controlling the expression from the encoding sequence is not induced, and when the cells reach a suitable density in the growth medium, the promoter can be induced for expression from the encoding sequence. Other control elements for efficient gene transcription or message translation are well known in the art to include enhancers, transcription or translation initiation signals, transcription termination and polyadenylation sequences, and the like.

### EXAMPLE 7

In another preferred embodiment, synthetic peptide according to the present invention further comprises a macromolecular carrier. Such macromolecular carriers are well known in the art to include, but are not limited to, serum proteins (the whole protein, or a substantial portion thereof), polymers, carbohydrates, and lipid-fatty acid conjugates, fatty acids, and the like. Serum proteins typically used as macromolecular carriers include, but are not limited to, transferrin, albumin, immunoglobulins (preferably IgG or one or more chains thereof), or hormones; wherein the protein is preferably human, and more preferably a recombinant human protein. Polymers typically used as macromolecular carriers include, but are not limited to, polylysines or poly(D-L-alanine)-poly(L-lysine)s, or polyols. A preferred polyol comprises a water-soluble poly(alkylene oxide) polymer, and can have a linear or branched chain. Suitable polyols include, but are not limited to, polyethylene glycol (PEG), polypropylene glycol (PPG), and PEG-PPG copolymers.

In one example, the macromolecular carrier may be conjugated to synthetic peptide. For example, in using a polyol, typically the polyol is derivatized or reacted with a coupling agent to form an “activated” polyol having one or more terminal reactive groups which can be used to react with a reactive functionality (e.g., preferably, a free amine group) of the synthetic peptide using methods standard in the art. Such reactive groups may include, but are not limited to, a hydroxy group, amino group, aldehyde group, and the like. The polyol used may comprise a linear chain or branched chain polymer. In another example, a synthetic peptide according to the present invention is synthesized, the last step of the synthesis process being the addition of a maleimide group (e.g., by a step in the solid phase synthesis of adding 3-maleimidoproprionic acid, washing, and then cleaving the synthetic peptide containing the maleimide group from the resin). Such methods are known in the art (see, e.g., WO 00/69902). The synthetic peptide may then be administered (preferably, parenterally) to an individual such that the synthetic peptide conjugates to a macromolecular carrier such as a blood component (preferably, a serum protein, and more preferably, albumin). In another example, recombinant human protein (e.g., albumin, transferrin, immunoglobulin, or the like) may be charged (“cationized”) and then thiolated using standard coupling agents known in the art (e.g., using N-succinimidyl S-acetylthio-acetate). The thiolated, charged recombinant human protein may be coupled to avidin using standard coupling reagents known in the art (using m-maleimidobenzoyl-N-hydroxysuccinimide ester). The resultant avidinylated human protein may then be reacted with synthetic peptide which had been previously biotinylated using methods standard in the art. Thus, the result is synthetic peptide that has been linked to macromolecular carrier.

In an alternative example, the macromolecular carrier may be genetically expressed with synthetic peptide; e.g., as part of a fusion protein. For example, a DNA sequence encoding albumin may be cloned into a nucleic acid molecule comprising a vector along with the DNA sequence encoding a linker and the DNA sequence encoding synthetic peptide according to the present invention, such that the resultant gene product is an albumin fusion protein comprising albumin with synthetic peptide linked at the C-terminal end, N-terminal end, or both the C-terminal and N-terminal ends of albumin. Such vectors and expression systems, preferably for yeast expression, are well known in the art (see, e.g., U.S. Pat. Nos. 5,728,553 & 5,965,386). Useful yeast plasmid vectors are generally commercially available (e.g., pRS403-406 series and pRS413-416 series) and which may incorporate the yeast selectable markers (e.g., his3, trp1, leu2, ura3, and the like). An expression vector, containing a polynucleotide encoding an albumin-synthetic peptide fusion protein for yeast expression, may comprise an expression cassette comprising: a yeast promoter (e.g., a Sacchromyces PRB1 promoter); a sequence encoding a secretion leader which will facilitate secretion of the expressed gene product (e.g., could be the natural human albumin secretion leader and/or a yeast-derived secretion leader); a sequence encoding human albumin (e.g., as disclosed in Genbank); a sequence encoding a linker (e.g., the linker comprising a stretch of 5-20 amino acids, and more preferably amino acids that include glycine and serine); a polynucleotide encoding synthetic peptide; and a transcription terminator (e.g., Saccharomyces ADH1). As apparent to one skilled in the art, if it is desired to have synthetic peptide being in the N-terminal region of the albumin fusion protein, then the polynucleotide encoding synthetic peptide would be placed between the promoter and the nucleic acid sequence encoding human albumin. The resultant nucleic acid molecule comprising an expression vector may then be used to transform yeast, and culture conditions for recombinant production, as well as purification of the recombinant product, could be performed using methods known in the art. Thus, obtained can be synthetic peptide further comprising a macromolecular carrier.

As an illustrative example, a fusion protein containing a synthetic peptide according to the present invention was produced. A nucleic acid molecule comprising an expression vector was constructed which contained a polynucleotide (SEQ ID NO:112) encoding a fusion protein (SEQ ID NO:113) comprising a maltose binding protein (“MBP”), a cleavable linker, and a synthetic peptide (SEQ ID NO:11) using standard methods known in the art. The resultant expression vector was transformed into an E. coli strain as the host expression system, and the transformed cells were grown and then induced to express the fusion protein by the addition of IPTG to the bacterial culture using standard methods known in the art. The induced bacterial cells were lysed with a microfluidizer, and the lysates were cleared of bacterial debris by centrifugation using standard methods known in the art. The clarified lysate was then subjected to chromatography using columns packed with amylose resin for binding to the fusion protein (via the MBP portion). The columns were then washed, followed by elution of the fusion protein with a solution containing maltose. The isolated fusion protein containing synthetic peptide, tested for antiviral activity using the methods outlined in Example 1, exhibited antiviral activity (e.g., an IC50 against HIV IIIB of <0.10 μg/ml) against HIV-1.

The foregoing description of the specific embodiments of the present invention have been described in detail for purposes of illustration. In view of the descriptions and illustrations, others skilled in the art can, by applying, current knowledge, readily modify and/or adapt the present invention for various applications without departing from the basic concept; and thus, such modifications and/or adaptations are intended to be within the meaning and scope of the appended claims.

What is claimed is

1. A synthetic peptide comprising an amino acid sequence derived from a base sequence: of one or more of SEQ ID NO:2, SEQ ID NO:3 or SEQ ID NO:4, wherein:
(a) the amino acid sequence of the synthetic peptide differs from amino acid sequence of the base sequence by substitution of between about 5% and about 60% of amino acids within the base sequence with:
(i) a helix-promoting amino acid, and
(ii) a charged amino acid residue, resulting in formation of a plurality of ion pairs absent in the base sequence; and
(b) the synthetic peptide demonstrates an improved biological activity, as compared to the base sequence, wherein the improved biological activity comprises antiviral activity comprising an IC50 of less than 0.50 μg/ml against an HIV strain that is resistant to the base sequence.

2. A synthetic peptide according to claim 1, wherein the synthetic peptide has an amino acid sequence consisting of any one of:
(a) SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:83, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, or (b) an amino acid sequence having at least 90% identity with any one or more of SEQ ID NOs:6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 88, 89, 90, 91, 92, 93, 94, 95, 96, 97, and 98.

3. A synthetic peptide according to claim 2, further comprising a component selected from the group consisting of one or more reactive functionalities, a pharmaceutically acceptable carrier, a macromolecular carrier, and a combination thereof.

4. A synthetic peptide according to claim 3, wherein the component is a macromolecular carrier which forms a fusion protein comprising the synthetic peptide.

5. A nucleic acid molecule comprising a nucleotide sequence encoding a synthetic peptide according to claim 2.

6. A nucleic acid molecule comprising a nucleotide sequence encoding a synthetic peptide according to claim 4.

7. A pharmaceutical composition comprising a synthetic peptide according to claim 2 and a pharmaceutically acceptable carrier.

8. A pharmaceutical composition comprising a synthetic peptide according to claim 3 and a pharmaceutically acceptable carrier.

9. A synthetic peptide having an amino acid sequence derived from a base sequence of one or more of SEQ ID NO:2, SEQ ID NO:3 or SEQ ID NO:4; wherein the synthetic peptide differs from the base sequence by between 2 and 5 amino acids comprising a helix-promoting amino acid; wherein the helix-promoting amino acids replace amino acids in the base sequence in positions corresponding to “a” and “d” positions of heptads within the base sequence; wherein the helix-promoting amino acids result in formation of from 1 to 3 additional leucine zipper-like motifs in the synthetic peptide, as compared to a number of leucine zipper-like motifs found in the base sequence; and wherein the synthetic peptide demonstrates an improved biological activity, as compared to the base sequence, wherein the improved biological activity comprises one or more of (a) an increase in antiviral activity against an HIV strain that is resistant to a base sequence, and (b) improved pharmacokinetic properties.

10. A synthetic peptide according to claim 9, wherein the synthetic peptide has an amino acid sequence consisting of any one of SEQ ID NO:5, SEQ ID NO:82, SEQ ID NO:84, SEQ ID NO:85, SEQ ID NO:86, and SEQ ID NO:87.

11. A synthetic peptide according to claim 9, further comprising a component selected from the group consisting of one or more reactive functionalities, a pharmaceutically acceptable carrier, a macromolecular carrier, and a combination thereof.

12. A synthetic peptide according to claim 10, further comprising a component selected from the group consisting of one or more reactive functionalities, a pharmaceutically acceptable carrier, a macromolecular carrier, and a combination thereof.

13. A synthetic peptide having an amino acid sequence consisting of any one of:
(a) SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, or SEQ ID NO:98; or
(b) an amino acid sequence having at least 90% identity with any one or more of SEQ ID NOs:6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 83, 88, 89, 90, 91, 92, 93, 94, 95, 96, 97, and 98 while still having
(i) an addition of a plurality of helix-promoting amino acids, as compared to corresponding amino acid positions within a base sequence of any one or more of SEQ ID NO: 2, SEQ ID NO:3, and SEQ ID NO:4, and
(ii) an addition of a plurality of charged amino acids as compared to corresponding amino acid positions within the base sequence replaced by the plurality of charged amino acids, in forming a plurality of ion pairs in the synthetic peptide which are absent in the base sequence; and
(iii) an improved biological activity, as compared to the base sequence.

14. A synthetic peptide according to claim 13, further comprising a component selected from the group consisting of one or more reactive functionalities, a pharmaceutically acceptable carrier, a macromolecular carrier, and a combination thereof.

15. A synthetic peptide according to claim 14, wherein the component is a macromolecular carrier which forms a fusion protein comprising the synthetic peptide.

16. A nucleic acid molecule comprising a nucleotide sequence encoding a synthetic peptide according to claim 13.

17. A nucleic acid molecule comprising a nucleotide sequence encoding a synthetic peptide according to claim 15.

18. A pharmaceutical composition comprising a synthetic peptide according to claim 13 and a pharmaceutically acceptable carrier.

19. A pharmaceutical composition comprising a synthetic peptide according to claim 14 and a pharmaceutically acceptable carrier.

20. A synthetic peptide having an amino acid sequence consisting of SEQ ID NO:97.

21. A synthetic peptide according to claim 20, further comprising a component selected from the group consisting of one or more reactive functionalities, a pharmaceutically acceptable carrier, a macromolecular carrier, and a combination thereof.

22. A synthetic peptide according to claim 21, wherein the component is a macromolecular carrier which forms a fusion protein comprising the synthetic peptide.

23. A nucleic acid molecule comprising a nucleotide sequence encoding a synthetic peptide according to claim 20.

24. A nucleic acid molecule comprising a nucleotide sequence encoding a synthetic peptide according to claim 22.

25. A pharmaceutical composition comprising a synthetic peptide according to claim 20 and a pharmaceutically acceptable carrier.

26. A pharmaceutical composition comprising a synthetic peptide according to claim 21 and a pharmaceutically acceptable carrier.

27. A therapeutic regimen comprising a combination of antiviral agents for treatment of HIV-1, the combination comprising a synthetic peptide according to claim 2, and one or more antiviral agents selected from the group consisting of an HIV entry inhibitor, HIV integrase inhibitor, reverse transcriptase inhibitor, protease inhibitor, vif-inhibitor, viral-specific transcription inhibitor, viral processing inhibitor, HIV maturation inhibitor, and a combination thereof.

28. A therapeutic regimen comprising a combination of antiviral agents for treatment of HIV-1, the combination comprising a synthetic peptide according to claim 3, and one or more antiviral agents selected from the group consisting of an HIV entry inhibitor, HIV integrase inhibitor, reverse transcriptase inhibitor, protease inhibitor, vif-inhibitor, viral-specific transcription inhibitor, viral processing inhibitor, HIV maturation inhibitor, and a combination thereof.

29. A therapeutic regimen comprising a combination of antiviral agents for treatment of HIV-1, the combination comprising a synthetic peptide according to claim 10, and one or more antiviral agents selected from the group consisting of an HIV entry inhibitor, HIV integrase inhibitor, reverse transcriptase inhibitor, protease inhibitor, vif-inhibitor, viral-specific transcription inhibitor, viral processing inhibitor, HIV maturation inhibitor, and a combination thereof.

30. A therapeutic regimen comprising a combination of antiviral agents for treatment of HIV-1, the combination comprising a synthetic peptide according to claim 12, and one or more antiviral agents selected from the group consisting of an HIV entry inhibitor, HIV integrase inhibitor, reverse transcriptase inhibitor, protease inhibitor, vif-inhibitor, viral-specific transcription inhibitor, viral processing inhibitor, HIV maturation inhibitor, and a combination thereof.

31. A therapeutic regimen comprising a combination of antiviral agents for treatment of HIV-1, the combination comprising a synthetic peptide according to claim 13, and one or more antiviral agents selected from the group consisting of an HIV entry inhibitor, HIV integrase inhibitor, reverse transcriptase inhibitor, protease inhibitor, vif-inhibitor, viral-specific transcription inhibitor, viral processing inhibitor, HIV maturation inhibitor, and a combination thereof.

32. A therapeutic regimen comprising a combination of antiviral agents for treatment of HIV-1, the combination comprising a synthetic peptide according to claim 14, and one or more antiviral agents selected from the group consisting of an HIV entry inhibitor, HIV integrase inhibitor, reverse transcriptase inhibitor, protease inhibitor, vif-inhibitor, viral-specific transcription inhibitor, viral processing inhibitor, HIV maturation inhibitor, and a combination thereof.

33. A therapeutic regimen comprising a combination of antiviral agents for treatment of HIV-1, the combination comprising a synthetic peptide according to claim 20, and one or more antiviral agents selected from the group consisting of an HIV entry inhibitor, HIV integrase inhibitor, reverse transcriptase inhibitor, protease inhibitor, vif-inhibitor, viral-specific transcription inhibitor, viral processing inhibitor, HIV maturation inhibitor, and a combination thereof.

34. A therapeutic regimen comprising a combination of antiviral agents for treatment of HIV-1, the combination comprising a synthetic peptide according to claim 21, and one or more antiviral agents selected from the group consisting of an HIV entry inhibitor, HIV integrase inhibitor, reverse transcriptase inhibitor, protease inhibitor, vif-inhibitor, viral-specific transcription inhibitor, viral processing inhibitor, HIV maturation inhibitor, and a combination thereof.

35. A method for inhibition of transmission of HIV to a cell, comprising contacting the virus, in the presence of a cell, with an amount of synthetic peptide according claim 2 effective to inhibit infection of the cell by HIV.

36. A method for inhibition of transmission of HIV to a cell, comprising contacting the virus, in the presence of a cell, with an amount of synthetic peptide according to claim 3 effective to inhibit infection of the cell by HIV.

37. A method for inhibition of transmission of HIV to a cell, comprising contacting the virus, in the presence of a cell, with an amount of synthetic peptide according to claim 10 effective to inhibit infection of the cell by HIV.

38. A method for inhibition of transmission of HIV to a cell, comprising contacting the virus, in the presence of a cell, with an amount of synthetic peptide according to claim 12 effective to inhibit infection of the cell by HIV.

39. A method for inhibition of transmission of HIV to a cell, comprising contacting the virus, in the presence of a cell, with an amount of synthetic peptide according to claim 13 effective to inhibit infection of the cell by HIV.

40. A method for inhibition of transmission of HIV to a cell, comprising contacting the virus, in the presence of a cell, with an amount of synthetic peptide according to claim 14 effective to inhibit infection of the cell by HIV.

41. A method for inhibition of transmission of HIV to a cell, comprising contacting the virus, in the presence of a cell, with an amount of synthetic peptide according to claim 20 effective to inhibit infection of the cell by HIV.

42. A method for inhibition of transmission of HIV to a cell, comprising contacting the virus, in the presence of a cell, with an amount of synthetic peptide according to claim 21 effective to inhibit infection of the cell by HIV.

43. A method for inhibiting HIV fusion, comprising contacting the virus, in the presence of a cell, with an amount of synthetic peptide according to claim 2 effective to inhibit HIV fusion.

44. A method for inhibiting HIV fusion, comprising contacting the virus, in the presence of a cell, with an amount of synthetic peptide according to claim 3 effective to inhibit HIV fusion.

45. A method for inhibiting HIV fusion, comprising contacting the virus, in the presence of a cell, with an amount of synthetic peptide according to claim 10 effective to inhibit HIV fusion.

46. A method for inhibiting HIV fusion, comprising contacting the virus, in the presence of a cell, with an amount of synthetic peptide according to claim 12 effective to inhibit HIV fusion.

47. A method for inhibiting HIV fusion, comprising contacting the virus, in the presence of a cell, with an amount of synthetic peptide according to claim 13 effective to inhibit HIV fusion.

48. A method for inhibiting HIV fusion, comprising contacting the virus, in the presence of a cell, with an amount of synthetic peptide according to claim 14 effective to inhibit HIV fusion.

49. A method for inhibiting HIV fusion, comprising contacting the virus, in the presence of a cell, with an amount of synthetic peptide according to claim 20 effective to inhibit HIV fusion.

50. A method for inhibiting HIV fusion, comprising contacting the virus, in the presence of a cell, with an amount of synthetic peptide according to claim 21 effective to inhibit HIV fusion.

51. A method for treating an HIV-infected individual comprising administering to the individual an amount of synthetic peptide according to claim 2 effective to achieve, in the treated individual, a therapeutic application selected from the group consisting of a reduction in the viral load of HIV, an increase in circulating CD4⁺ cell population, and a combination thereof.

52. A method for treating an HIV-infected individual comprising administering to the individual an amount of synthetic peptide according to claim 3 effective to achieve, in the treated individual, a therapeutic application selected from the group consisting of a reduction in the viral load of HIV, an increase in circulating CD4⁺ cell population, and a combination thereof.

53. A method for treating an HIV-infected individual comprising administering to the individual an amount of synthetic peptide according to claim 10 effective to achieve, in the treated individual, a therapeutic application selected from the group consisting of a reduction in the viral load of HIV, an increase in circulating CD4⁺ cell population, and a combination thereof.

54. A method for treating an HIV-infected individual comprising administering to the individual an amount of synthetic peptide according to claim 12 effective to achieve, in the treated individual, a therapeutic application selected from the group consisting of a reduction in the viral load of HIV, an increase in circulating CD4⁺ cell population, and a combination thereof.

55. A method for treating an HIV-infected individual comprising administering to the individual an amount of synthetic peptide according to claim 13 effective to achieve, in the treated individual, a therapeutic application selected from the group consisting of a reduction in the viral load of HIV, an increase in circulating CD4⁺ cell population, and a combination thereof.

56. A method for treating an HIV-infected individual comprising administering to the individual an amount of synthetic peptide according to claim 14 effective to achieve, in the treated individual, a therapeutic application selected from the group consisting of a reduction in the viral load of HIV, an increase in circulating CD4⁺ cell population, and a combination thereof.

57. A method for treating an HIV-infected individual comprising administering to the individual an amount of synthetic peptide according to claim 20 effective to achieve, in the treated individual, a therapeutic application selected from the group consisting of a reduction in the viral load of HIV, an increase in circulating CD4⁺ cell population, and a combination thereof.

58. A method for treating an HIV-infected individual comprising administering to the individual an amount of synthetic peptide according to claim 21 effective to achieve, in the treated individual, a therapeutic application selected from the group consisting of a reduction in the viral load of HIV, an increase in circulating CD4⁺ cell population, and a combination thereof.

59. The method according to claim 51, wherein the synthetic peptide is administered as an antiviral agent in a therapeutic regimen comprising a combination of antiviral agents used to treat HIV infection.

60. The method according to claim 52, wherein the synthetic peptide is administered as an antiviral agent in a therapeutic regimen comprising a combination of antiviral agents used to treat HIV infection.

61. The method according to claim 53, wherein the synthetic peptide is administered as an antiviral agent in a therapeutic regimen comprising a combination of antiviral agents used to treat HIV infection.

62. The method according to claim 54, wherein the synthetic peptide is administered as an antiviral agent in a therapeutic regimen comprising a combination of antiviral agents used to treat HIV infection.

63. The method according to claim 55, wherein the synthetic peptide is administered as an antiviral agent in a therapeutic regimen comprising a combination of antiviral agents used to treat HIV infection.

64. The method according to claim 56, wherein the synthetic peptide is administered as an antiviral agent in a therapeutic regimen comprising a combination of antiviral agents used to treat HIV infection.

65. The method according to claim 57, wherein the synthetic peptide is administered as an antiviral agent in a therapeutic regimen comprising a combination of antiviral agents used to treat HIV infection.

66. The method according to claim 58, wherein the synthetic peptide is administered as an antiviral agent in a therapeutic regimen comprising a combination of antiviral agents used to treat HIV infection.


SEQUENCE LISTING (113 sequences)

SEQ ID NO: 1 - protein, 36 aa, Artificial
  synthesized
YTSLIHSLIEESQNQQEKNEQELLELDKWASLWNWF

SEQ ID NO: 2 - protein, 36 aa, Artificial
  synthesized
MTWMEWDREINNYTSLIHSLIEESQNQQEKNEQELL

SEQ ID NO: 3 - protein, 36 aa, Artificial
  synthesized
WMEWDREINNYTSLIHSLIEESQNQQEKNEQELLEL

SEQ ID NO: 4 - protein, 38 aa, Artificial
  synthesized
MTWMEWDREINNYTSLIHSLIEESQNQQEKNEQELLEL

SEQ ID NO: 5 - protein, 38 aa, Artificial
  synthesized
MTWMAWDRAIANYAALIHALIEAAQNQQEKNEAALLEL

SEQ ID NO: 6 - protein, 38 aa, Artificial
  synthesized
TTWEAWDRAIAEAAARIEALIRAAQEQQEKNEAALREL

SEQ ID NO: 7 - protein, 38 aa, Artificial
  synthesized
TTWEAWDRAIAEYAARAEALIRAAQEQQEKNEAALREL

SEQ ID NO: 8 - protein, 38 aa, Artificial
  synthesized
MTWEAWDRAIAEYAARIEALLRAAQEQQEKNEAALREL

SEQ ID NO: 9 - protein, 46 aa, Artificial
  synthesized
APKEMTWEAWDRAIAEYAARIEALIRAAQEQQEKNEAALRELKQGI

SEQ ID NO: 10 - protein, 38 aa, Artificial
  synthesized
MTWEAWDRAIAEYAARIEALIRAAQEQQEKNEAALREL

SEQ ID NO: 11 - protein, 38 aa, Artificial
  synthesized
TTWEAWDRAIAEYAARIEALIRAAQEQQEKNEAALREL

SEQ ID NO: 12 - protein, 38 aa, Artificial
  synthesized
MTWEAWDRAIAEAAARIEALIRAAQEQQEKNEAALREL

SEQ ID NO: 13 - protein, 38 aa, Artificial
  synthesized
MTWEAWDRAIAEYAARIEALIRAAQEQQEKAEAALREL

SEQ ID NO: 14 - protein, 42 aa, Artificial
  synthesized
MTWEAWDRAIAEYAARIEALIRAAQEQQEKNEAALRELWEWF

SEQ ID NO: 15 - protein, 42 aa, Artificial
  synthesized
TTWEAWDRAIAEYAARIEALIRAAQEQQEKNEAALRELWEWF

SEQ ID NO: 16 - protein, 41 aa, Artificial
  synthesized
TTWEAWDRAIAEYAARIEALIRAAQEQQEKNEAALREWEWF

SEQ ID NO: 17 - protein, 38 aa, Artificial
  synthesized
TTWEAWDRAIAEYAARIEALIRAAQEQQEKNEAAWEWF

SEQ ID NO: 18 - protein, 48 aa, Artificial
  synthesized
WEWFGGSGGSTTWEAWDRAIAEYAARIEALIRAAQEQQEKNEAALREL

SEQ ID NO: 19 - protein, 48 aa, Artificial
  synthesized
TTWEAWDRAIAEYAARIEALIRAAQEQQEKNEAALRELGGSGGSWEWF

SEQ ID NO: 20 - protein, 45 aa, Artificial
  synthesized
TTWEAWDRAIAEYAARIEALIRAAQEQQEKNEAALRELGGSGGSW

SEQ ID NO: 21 - protein, 45 aa, Artificial
  synthesized
WGGSGGSTTWEAWDRAIAEYAARIEALIRAAQEQQEKNEAALREL

SEQ ID NO: 22 - protein, 39 aa, Artificial
  synthesized
PTTWEAWDRAIAEYAARIEALIRAAQEQQEKNEAALREL

SEQ ID NO: 23 - protein, 40 aa, Artificial
  synthesized
PTTWEAWDRAIAEYAARIEALIRAAQEQQEKNEAALRELP

SEQ ID NO: 24 - protein, 38 aa, Artificial
  synthesized
TTWEAWDKAIAEYAAKIEALIKAAQEQQEKNEAALKEL

SEQ ID NO: 25 - protein, 38 aa, Artificial
  synthesized
TTWEAWDRAWQEWEQKIEALIRAAQEQQEKNEAALREL

SEQ ID NO: 26 - protein, 38 aa, Artificial
  synthesized
TTWAAWDAAIAEYAARIEALIRAAQEQQEKNEAALREL

SEQ ID NO: 27 - protein, 38 aa, Artificial
  synthesized
TTWEAWDRAIAAYAAAIEALIRAAQEQQEKNEAALREL

SEQ ID NO: 28 - protein, 38 aa, Artificial
  synthesized
TTWEAWDRAIAEYAARIAALIAAAQEQQEKNEAALREL

SEQ ID NO: 29 - protein, 38 aa, Artificial
  synthesized
TTWEAWDRAIAEYAARIEALIRAAQAQQEANEAALREL

SEQ ID NO: 30 - protein, 38 aa, Artificial
  synthesized
TTWEAWDRAIAEYAARIEALIRAAQEQQEKNAAALAEL

SEQ ID NO: 31 - protein, 38 aa, Artificial
  synthesized
TTWEEWDREINEYAARIEALIRAAQEQQEKNEAALREL

SEQ ID NO: 32 - protein, 38 aa, Artificial
  synthesized
TTWEAWDRAIAEYTSRIESLIRAAQEQQEKNEAALREL

SEQ ID NO: 33 - protein, 38 aa, Artificial
  synthesized
TTWEAWDRAIAEYAARIEALIRAAQNQQEKNEAALREL

SEQ ID NO: 34 - protein, 38 aa, Artificial
  synthesized
TTWEAWDRAIAEYAARIEALIRAAQEQQEKNEAALLEL

SEQ ID NO: 35 - protein, 38 aa, Artificial
  synthesized
TTWEAWDRAIAEYAARIHALIEAAQEQQEKNEAALREL

SEQ ID NO: 36 - protein, 38 aa, Artificial
  synthesized
TTWEAWDRAIANYAALIEALIRAAQEQQEKNEAALREL

SEQ ID NO: 37 - protein, 48 aa, Artificial
  synthesized
AKEAAQRANATTWEAWDRAIAEYAARIEALIRAAQEQQEKNEAALREL

SEQ ID NO: 38 - protein, 48 aa, Artificial
  synthesized
NKELEQRWNNTTWEAWDRAIAEYAARIEALIRAAQEQQEKNEAALREL

SEQ ID NO: 39 - protein, 48 aa, Artificial
  synthesized
EKAARQAENAARWEAWDRAIAEYAARIEALIRAAQEQQEKNEAALREL

SEQ ID NO: 40 - protein, 48 aa, Artificial
  synthesized
EKSLRQIENNTRWEAWDRAIAEYAARIEALIRAAQEQQEKNEAALREL

SEQ ID NO: 41 - protein, 48 aa, Artificial
  synthesized
TTWEAWDRAIAEYAARIEALIRAAQEQQEKNEAALRELAAREAAWRWF

SEQ ID NO: 42 - protein, 48 aa, Artificial
  synthesized
TTWEAWDRAIAEYAARIEALIRAAQEQQEKNEAALRELDKREALWRWF

SEQ ID NO: 43 - protein, 48 aa, Artificial
  synthesized
TTWEAWDRAIAEYAARIEALIRAAQEQQEKNEAALRELDKRESLWRWF

SEQ ID NO: 44 - protein, 49 aa, Artificial
  synthesized
GAKEAAQRANATTWEAWDRAIAEYAARIEALIRAAQEQQEKNEAALREL

SEQ ID NO: 45 - protein, 49 aa, Artificial
  synthesized
GEKAARQAENAARWEAWDRAIAEYAARIEALIRAAQEQQEKNEAALREL

SEQ ID NO: 46 - protein, 37 aa, Artificial
  synthesized
TTWEAWDRAIAEYAARIEALIRAAQEQQEKNEAALRE

SEQ ID NO: 47 - protein, 36 aa, Artificial
  synthesized
TTWEAWDRAIAEYAARIEALIRAAQEQQEKNEAALR

SEQ ID NO: 48 - protein, 35 aa, Artificial
  synthesized
TTWEAWDRAIAEYAARIEALIRAAQEQQEKNEAAL

SEQ ID NO: 49 - protein, 33 aa, Artificial
  synthesized
TTWEAWDRAIAEYAARIEALIRAAQEQQEKNEA

SEQ ID NO: 50 - protein, 38 aa, Artificial
  synthesized
TTWEAWDRAIAEYAARIEALIRAAQEQQEKNEAILREL

SEQ ID NO: 51 - protein, 38 aa, Artificial
  synthesized
TTWEAWDRAIAEYAARIEALIRALQEQQEKNEAALREL

SEQ ID NO: 52 - protein, 38 aa, Artificial
  synthesized
TTWEAWDRAIAEYAARIEALIRALQEQQEKNEAILREL

SEQ ID NO: 53 - protein, 38 aa, Artificial
  synthesized
TTWEAWDRAIAEYAARIEALIRAAQELQEKNEAALREL

SEQ ID NO: 54 - protein, 38 aa, Artificial
  synthesized
TTWEAWDRAIAEYAARIEALIRAAQEQQEKLEAALREL

SEQ ID NO: 55 - protein, 38 aa, Artificial
  synthesized
TTWEAWDRAAAEYAARIEALIRAAQEQQEKNEAALREL

SEQ ID NO: 56 - protein, 38 aa, Artificial
  synthesized
TTWEAWDRAIAEYAARIEAAIRAAQEQQEKNEAALREL

SEQ ID NO: 57 - protein, 38 aa, Artificial
  synthesized
TTWEAWDRAIAEYAARIEALIRAAQEQQEKNEAALREA

SEQ ID NO: 58 - protein, 36 aa, Artificial
  synthesized
WEAWDRAIAEYAARIEALIRAAQEQQEKNEAALREL

SEQ ID NO: 59 - protein, 37 aa, Artificial
  synthesized
WEAWDRAIAEYAARIEALIRAAQEQQEKNEAALRELA

SEQ ID NO: 60 - protein, 38 aa, Artificial
  synthesized
TTWEAWDRAIAEYAARIEALIRAAQEAQEKNEAALREL

SEQ ID NO: 61 - protein, 38 aa, Artificial
  synthesized
ETWKEWDRAIEEYKKRIEELIKAAENQQEKNKEALREL

SEQ ID NO: 62 - protein, 38 aa, Artificial
  synthesized
MAWMEWDRRIEAYARLIAELIARAQEQQEKNEAALREL

SEQ ID NO: 63 - protein, 41 aa, Artificial
  synthesized
TTWEAWDRAIAEYAARIEALIRAAQEQQEKNEQQLREWEWF

SEQ ID NO: 64 - protein, 41 aa, Artificial
  synthesized
TTWEAWDRAIAEYAARIEALIRAAQEQQEKNEAALREWEWI

SEQ ID NO: 65 - protein, 38 aa, Artificial
  synthesized
TTWDAWDRAIADYAARIDALIRAAQDQQEKNDAALREL

SEQ ID NO: 66 - protein, 41 aa, Artificial
  synthesized
TTWEAWDRAIAEYAARIEALIRAAQEQQEKAEAALREWEWF

SEQ ID NO: 67 - protein, 38 aa, Artificial; identical to SEQ ID NO: 25
  synthesized

SEQ ID NO: 68 - protein, 52 aa, Artificial
  synthesized
WASLWEWFGGSGGSTTWEAWDRAIAEYAARIEALIRAAQEQQEKNEAALREL

SEQ ID NO: 69 - protein, 52 aa, Artificial
  synthesized
TTWEAWDRAIAEYAARIEALIRAAQEQQEKNEAALRELGGSGGSWASLWEWF

SEQ ID NO: 70 - protein, 38 aa, Artificial
  synthesized
TTWEAWDRAIAEYAARIEALIRAAQEQQEKNEQELREL

SEQ ID NO: 71 - protein, 38 aa, Artificial; identical to SEQ ID NO: 60
  synthesized

SEQ ID NO: 72 - protein, 41 aa, Artificial
  synthesized
TTWEAWDRAIAEYAARIEALIRAAQEQQEKNEAALREWWWW

SEQ ID NO: 73 - protein, 47 aa, Artificial
  synthesized
TTWEAWDRAIAEYAARIEALIRAAQEQQEKNEAALRELDKWSLWRWF

SEQ ID NO: 74 - protein, 47 aa, Artificial
  synthesized
TTWEAWDRAIAEYAARIEALIRAAQEQQEKNEAALRALDKWEALWRF

SEQ ID NO: 75 - protein, 41 aa, Artificial
  synthesized
TTWEAWDRAWQEWEQKIEALIRAAQEQQEKNEAALREWEWF

SEQ ID NO: 76 - protein, 38 aa, Artificial
  synthesized
LTWEAWDRAIAEYAARIEALIRAAQEQQEKNEAALREL

SEQ ID NO: 77 - protein, 38 aa, Artificial
  synthesized
TTWMAWDRAIAEYAARIEALIRAAQEQQEKNEAALREL

SEQ ID NO: 78 - protein, 55 aa, Artificial
  synthesized
TTWEAWDRAIAEYAARIEALIRAAQEQQEKNEAALRELGGSGGSGGSWASLWEWF

SEQ ID NO: 79 - protein, 38 aa, Artificial; identical to SEQ ID NO: 6
  synthesized

SEQ ID NO: 80 - protein, 59 aa, Artificial
  synthesized
GAKEAAQRANATTWEAWDRAIAEYAARIEALIRAAQEQQEKNEAALRELDKWASLWNWF

SEQ ID NO: 81 - protein, 39 aa, Artificial
  synthesized
PANWKAWEAQIQKYQRQIAELIANAKKQQEQNEKALREL

SEQ ID NO: 82 - protein, 38 aa, Artificial
  synthesized
MTWMEWDREINNYTSLIHSLIEEIQNQQEKNEQELLEL

SEQ ID NO: 83 - protein, 38 aa, Artificial
  synthesized
TTWEEWDREINEYTSRIESLIRAAQEQQEKNAAALAEL

SEQ ID NO: 84 - protein, 38 aa, Artificial
  synthesized
MTWMEWDREINNYTSLIHSLIEEIQNIQEKNEQELLEL

SEQ ID NO: 85 - protein, 38 aa, Artificial
  synthesized
MTWMEWDREINNYTSLIHSLIEEIQNIQEKIEQELLEL

SEQ ID NO: 86 - protein, 38 aa, Artificial
  synthesized
MTWMEWDREINNYTSLIHSLIEEIQNIQEKNEQILLEL

SEQ ID NO: 87 - protein, 38 aa, Artificial
  synthesized
MTWMEWDREINNYTSLIHSLIEEAQNQQEKNEQALLEL

SEQ ID NO: 88 - protein, 42 aa, Artificial
  synthesized
PANWKAWEAQIQKYQRQIAELIANAKKQQEQNEKALREWEWF

SEQ ID NO: 89 - protein, 38 aa, Artificial
  synthesized
ANWKAWEAQIQKYQRQIAELIANAKKQQEQNEKALREL

SEQ ID NO: 90 - protein, 38 aa, Artificial
  synthesized
TTWEAWDRAIAEYAARIEALIRAAQEQQEKNEAVLREL

SEQ ID NO: 91 - protein, 38 aa, Artificial
  synthesized
TTWEAWDRAIAEYAARIEALIRAAQEQQEKVEAALREL

SEQ ID NO: 92 - protein, 38 aa, Artificial
  synthesized
TTWEAWDRAIAEYAARIEALIRAAQEQQEKIEAALREL

SEQ ID NO: 93 - protein, 38 aa, Artificial
  synthesized
TTWEAWDRAIAEYAARIEALIRAAQELQEKNEAILREL

SEQ ID NO: 94 - protein, 38 aa, Artificial
  synthesized
TTWEAWDRAIAEYAARIEALIRALQELQEKNEAALREL

SEQ ID NO: 95 - protein, 38 aa, Artificial
  synthesized
TTWEAWDRAIAEYAARIEALIRAAQEQQEKNEALLREL

SEQ ID NO: 96 - protein, 38 aa, Artificial
  synthesized
TTWEAWDRAIAEYAARIEALLRAAQEQQEKNEAALREL

SEQ ID NO: 97 - protein, 38 aa, Artificial
  synthesized
TTWEAWDRAIAEYAARIEALLRALQEQQEKNEAALREL

SEQ ID NO: 98 - protein, 38 aa, Artificial
  synthesized
TTWEAWDRAIAEYAARIEALLRAAQEQQEKLEAALREL

SEQ ID NO: 99 - protein, 38 aa, Artificial
  synthesized
TTWEAWDRAIAEYAARIEALIRALQEQQEKLEAALREL

SEQ ID NO: 100 - protein, 38 aa, Artificial
  synthesized
TTWEAWDRAIAEYAARIEALIRAIQEQQEKLEAALREL

SEQ ID NO: 101 - protein, 38 aa, Artificial
  synthesized
TTWEAWDRAIAEYAARIEALIRALQEQQEKIEAALREL

SEQ ID NO: 102 - protein, 38 aa, Artificial
  synthesized
TTWEAWDRAIAEYAARIEALLRAIQEQQEKNEAALREL

SEQ ID NO: 103 - protein, 38 aa, Artificial
  synthesized
TTWEAWDRAIAEYAARIEALLRAAQEQQEKIEAALREL

SEQ ID NO: 104 - protein, 70 aa, Artificial
  synthesized
XAAXAAXAAEAXAADRAZAAAEXAAAARZAAEAZAAZAARABAAXAAEXAAXAAEKBAAEAAZAAREZAA

SEQ ID NO: 105 - protein, 34 aa, Artificial
  synthesized
WMEWDRKIEEYTKKIKKLIEESQEQQEKNEKELK

SEQ ID NO: 106 - protein, 34 aa, Artificial
  synthesized
WMEWDRKIEEYTKKIEELIKKSQEQQEKNEKELK

SEQ ID NO: 107 - protein, 35 aa, Artificial
  synthesized
WEEWDKKIEEYTKKIEELIKKSEEQQKKNEEELKK

SEQ ID NO: 108 - protein, 36 aa, Artificial
  synthesized
SWETWEREIENYTKQIYKILEESQEQQDRNEKDLLE

SEQ ID NO: 109 - DNA, 108 bp, Artificial
  synthesized
atgacctggatggagtgggacagagaaattaacaattacacaagcttaatacactccttaattgaagaatcgcaaaaccagcaagaaaagaatgaacaagaattattg

SEQ ID NO: 110 - DNA, 114 bp, Artificial
  synthesized
atgacctggatggcttgggacagagctattgctaattacgctgctttaatacacgctttaattgaagctgctcaaaaccagcaagaaaagaatgaagctgctttattggaatta

SEQ ID NO: 111 - DNA, 114 bp, Artificial
  synthesized
accacctgggaagcttgggacagagctattgctgaatacgctgctagaatagaagctttaattagagctgctcaagaacagcaagaaaagaatgaagctgctttaagagaatta

SEQ ID NO: 112 - DNA, 1338 bp, Artificial
  synthesized
atgaaaactgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcgaacaacaacaacaataacaataacaacaacctcgggatcgagggaaggatcccaacgaccgaaaacctgtattttcagggcgctaaagaagctgctcagcgtgctaacgctaccacctgggaagcttgggaccgtgctatcgctgaatacgctgctcgtatcgaagctctgatccgtgctgctcaggaacagcaggaaaaaaacgaagctgctctgcgtgaactg

SEQ ID NO: 113 - protein, 446 aa, Artificial
  synthesized
MKTEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGRIPTTENLYFQGAKEAAQRANATTWEAWDRAIAEYAARIEALIRAAQEQQEKNEAALREL